(19) United States
(12) Patent Application Publication
(10) Pub. No.: US 2024/0409897 A1
(43) Pub. Date: Dec. 12, 2024

(54) METHODS FOR AGGREGATING CELLS IN SUSPENSION

(71) Applicant: Satellite Biosciences, Inc., Cambridge, MA (US)

(72) Inventors: Christopher Garrison WILSON, Auburndale, MA (US); Marcus LEHMANN, Boston, MA (US); Julie MORSE, Acton, MA (US); Bridget ZHOU, Cambridge, MA (US); Amanda CHEN, Cambridge, MA (US); Peter MOUA, Boston, MA (US); Fabiola MUNARIN, Smithfield, RI (US); Joseph E. MARTURANO, Bedford, MA (US)

(21) Appl. No.: 18/704,357

(22) PCT Filed: Oct. 25, 2022
(86) PCT No.: PCT/US2022/047760

§ 371 Date: Apr. 24, 2024

Related U.S. Application Data
(60) Provisional application No. 63/271,662, filed on Oct. 25, 2021.
(60) Provisional application No. 63/346,575, filed on May 27, 2022.

(51) Int. Cl. C12N 5/077; C12N 5/071; C12N 11/04

(52) U.S. Cl. CPC C12N 5/0656; C12N 5/0671; C12N 5/0677; C12N 11/04; C12N 2501/39; C12N 2501/727; C12N 2513/00; C12N 2533/52; C12N 2533/56

(57) ABSTRACT

The invention features a method of producing aggregates of a plurality of cell populations (e.g., a first population of cells and a second population of cells). The method includes agitating a liquid medium that contains the cells in a bioreactor. These aggregates may be used for subsequent downstream applications, such as encapsulation in a biocompatible scaffold to form an engineered tissue construct.

### BACKGROUND OF THE INVENTION

Many diseases result from damage, malfunction, or loss of a single organ or tissue type. While certain strategies such as organ transplants can be effective, the demand for replacement organs is great. Tissue therapeutics, including the development of engineered tissue constructs (e.g., cell-based implants), are among the most promising multidisciplinary approaches to fulfill this demand. However, despite significant advances in the fields of cell biology, microfluidics, and engineering, to date, conventional approaches have failed to re-create functional tissues at a scale necessary to impart therapeutic efficacy. Formation of cell aggregates to generate tissues is critical as a first step towards creating useful tissues. However, mimicking biological conditions to achieve such conditions remains challenging. Accordingly, new methods for forming cell aggregates are needed.

### SUMMARY OF THE INVENTION

In one aspect, the invention features a method of producing aggregates of a plurality of cell populations that includes a first population of cells and a second population of cells. The method includes the step of (a) agitating a liquid medium that contains the first population of cells and the second population of cells in a bioreactor for a duration sufficient to form aggregates that include the first population of cells and the second population of cells. The first population of cells and the second population of cells are in suspension in the liquid medium during the agitating step. The method produces aggregates of the first population of cells and the second population of cells. The method may further include the step of (b) collecting the aggregates that include the first population of cells and the second population of cells.

In some embodiments, the first population of cells includes stromal cells (e.g., fibroblasts). The fibroblasts may be, e.g., primary fibroblasts, induced pluripotent stem cell (iPSC)-derived fibroblasts, or embryonic stem cell (ESC)-derived fibroblasts. In some embodiments, the fibroblasts are genetically engineered fibroblasts.

In some embodiments, the second population of cells includes parenchymal cells. The parenchymal cells may be, e.g., hepatocytes (e.g., human hepatocytes, e.g., primary human hepatocytes) or hepatocyte precursor cells. The hepatocytes may be, e.g., primary hepatocytes (e.g., primary human hepatocytes), iPSC-derived hepatocytes, or ESC-derived hepatocytes. The hepatocytes may be genetically engineered hepatocytes. The parenchymal cells may include pancreatic cells (e.g., alpha, beta, gamma, delta, or epsilon cells, or a combination thereof) or pancreatic precursor cells. The pancreatic cells may be, e.g., primary human pancreatic cells, iPSC-derived pancreatic cells, or ESC-derived pancreatic cells. In some embodiments, the pancreatic cells are genetically engineered pancreatic cells.

In some embodiments, the first population of cells includes fibroblasts, and the second population of cells includes hepatocytes.

In some embodiments, the first population of cells includes fibroblasts, and the second population includes hepatocyte precursor cells.

In some embodiments, the first population of cells includes fibroblasts, and the second population includes pancreatic beta cells.

In some embodiments, one of the populations of cells includes endocrine, exocrine, paracrine, heterocrine, autocrine, or juxtacrine cells.

In some embodiments, one of the populations of cells includes Leydig cells, adrenal cortical cells, pituitary cells, thyrocytes, granulosa cells, mammary gland epithelial cells, thymocytes, thymic epithelial cells, hypothalamus cells, skeletal muscle cells, smooth muscle cells, and/or neuronal cells. In some embodiments, the pituitary cells include thyrotropic pituitary cells, lactotropic pituitary cells, corticotropic pituitary cells, somatotropic pituitary cells, and/or gonadotropic pituitary cells. In some embodiments, the neuronal cells include dopaminergic cells.

In some embodiments, one of the populations of cells includes parenchymal cells (e.g., hepatocytes, pancreatic exocrine cells, myocytes, pancreatic endocrine cells, neurons, enterocytes, adipocytes, splenic cells, kidney cells, biliary cells, Kupffer cells, stellate cells, cardiac muscle cells, alveolar cells, bronchiolar cells, club cells, urothelial cells, mucous cells, parietal cells, chief cells, G cells, goblet cells, enteroendocrine cells, Paneth cells, M cells, tuft cells, glial cells, gall bladder cells, keratinocytes, melanocytes, Merkel cells, Langerhans cells, osteocytes, osteoclasts, esophageal cells, photoreceptor cells, and corneal epithelial cells). In some embodiments, the parenchymal cells are pancreatic cells (e.g., alpha, beta, gamma, delta, epsilon cells, or any combination thereof). In some embodiments, the parenchymal cells include beta cells.

In some embodiments, the cells are engineered cells, primary cells, or transdifferentiated cells. In some embodiments, a ratio of the first population of cells (e.g., stromal cells, e.g., fibroblasts) to the second population of cells (e.g., parenchymal cells, e.g., hepatocytes, e.g., primary human hepatocytes) is from 10:1 to 1:10, 5:1 to 1:5, 3:1 to 1:3, or 2:1 to 1:2 (e.g., 10:1 to 1:1, 9:1 to 1:1, 8:1 to 1:1, 7:1 to 1:1, 6:1 to 1:1, 5:1 to 1:1, 4:1 to 1:1, 3:1 to 1:1, 2:1 to 1:1, 1:10 to 1:1 to 1:2, 1:1 to 1:3, 1:1 to 1:4, 1:1 to 1:5, 1:1 to 1:6, 1:1 to 1:7, 1:1 to 1:8, 1:1 to 1:9, or 1:1 to 1:10). For example, the ratio of the first population of cells to the second population of cells may be 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10. In some embodiments, the ratio is 2:1.

In some embodiments, the first population of cells are present at a density of 1×10⁴ cells/mL to 1×10⁸ cells/mL. For example, the first population of cells may be present at a density of 1×10⁴ cells/mL to 1×10⁵ cells/mL (e.g., 1×10⁴ cells/mL, 2×10⁴ cells/mL, 3×10⁴ cells/mL, 4×10⁴ cells/mL, 5×10⁴ cells/mL, 6×10⁴ cells/mL, 7×10⁴ cells/mL, 8×10⁴ cells/mL, 9×10⁴ cells/mL, or 1×10⁵ cells/mL), 1×10⁵ cells/mL to 1×10⁶ cells/mL (e.g., 1×10⁵ cells/mL, 2×10⁵ cells/mL, 3×10⁵ cells/mL, 4×10⁵ cells/mL, 5×10⁵ cells/mL, 6×10⁵ cells/mL, 7×10⁵ cells/mL, 8×10⁵ cells/mL, 9×10⁵ cells/mL, or 1×10⁶ cells/mL), 1×10⁶ cells/mL to 1×10⁷ cells/mL (e.g., 1×10⁶ cells/mL, 2×10⁶ cells/mL, 3×10⁶ cells/mL, 4×10⁶ cells/mL, 5×10⁶ cells/mL, 6×10⁶ cells/mL, 7×10⁶ cells/mL, 8×10⁶ cells/mL, 9×10⁶ cells/mL, or 1×10⁷ cells/mL), or 1×10⁷ cells/mL to 1×10⁸ cells/mL (e.g., 1×10⁷ cells/mL, 2×10⁷ cells/mL, 3×10⁷ cells/mL, 4×10⁷ cells/mL, 5×10⁷ cells/mL, 6×10⁷ cells/mL, 7×10⁷ cells/mL, 8×10⁷ cells/mL, 9×10⁷ cells/mL, or 1×10⁸ cells/mL).

In some embodiments, the first population of cells includes fibroblasts (e.g., primary fibroblasts, e.g., primary human fibroblasts) and the density of the fibroblasts is 1×10⁵ cells/mL to 1×10⁷ cells/mL (e.g., 1×10⁵ cells/mL, 2×10⁵ cells/mL, 3×10⁵ cells/mL, 4×10⁵ cells/mL, 5×10⁵ cells/mL, 6×10⁵ cells/mL, 7×10⁵ cells/mL, 8×10⁵ cells/mL, 9×10⁵ cells/mL, 1×10⁶ cells/mL, 2×10⁶ cells/mL, 3×10⁶ cells/mL, 4×10⁶ cells/mL, 5×10⁶ cells/mL, 6×10⁶ cells/mL, 7×10⁶ cells/mL, 8×10⁶ cells/mL, 9×10⁶ cells/mL, or 1×10⁷ cells/mL). For example, the density of the fibroblasts may be, e.g., 6×10⁵ cells/mL.

In some embodiments, the second population of cells are present at a density of 1×10⁴ cells/mL to 1×10⁸ cells/mL. For example, the second population of cells may be present at a density of 1×10⁴ cells/mL to 1×10⁵ cells/mL (e.g., 1×10⁴ cells/mL, 2×10⁴ cells/mL, 3×10⁴ cells/mL, 4×10⁴ cells/mL, 5×10⁴ cells/mL, 6×10⁴ cells/mL, 7×10⁴ cells/mL, 8×10⁴ cells/mL, 9×10⁴ cells/mL, or 1×10⁵ cells/mL), 1×10⁵ cells/mL to 1×10⁶ cells/mL (e.g., 1×10⁵ cells/mL, 2×10⁵ cells/mL, 3×10⁵ cells/mL, 4×10⁵ cells/mL, 5×10⁵ cells/mL, 6×10⁵ cells/mL, 7×10⁵ cells/mL, 8×10⁵ cells/mL, 9×10⁵ cells/mL, or 1×10⁶ cells/mL), 1×10⁶ cells/mL to 1×10⁷ cells/mL (e.g., 1×10⁶ cells/mL, 2×10⁶ cells/mL, 3×10⁶ cells/mL, 4×10⁶ cells/mL, 5×10⁶ cells/mL, 6×10⁶ cells/mL, 7×10⁶ cells/mL, 8×10⁶ cells/mL, 9×10⁶ cells/mL, or 1×10⁷ cells/mL), or 1×10⁷ cells/mL to 1×10⁸ cells/mL (e.g., 1×10⁷ cells/mL, 2×10⁷ cells/mL, 3×10⁷ cells/mL, 4×10⁷ cells/mL, 5×10⁷ cells/mL, 6×10⁷ cells/mL, 7×10⁷ cells/mL, 8×10⁷ cells/mL, 9×10⁷ cells/mL, or 1×10⁸ cells/mL).

In some embodiments, the second population of cells includes hepatocytes (e.g., primary hepatocytes, e.g., primary human hepatocytes) and the density of the hepatocytes is 1×10⁵ cells/mL to 1×10⁷ cells/mL (e.g., 1×10⁵ cells/mL, 2×10⁵ cells/mL, 3×10⁵ cells/mL, 4×10⁵ cells/mL, 5×10⁵ cells/mL, 6×10⁵ cells/mL, 7×10⁵ cells/mL, 8×10⁵ cells/mL, 9×10⁵ cells/mL, 1×10⁶ cells/mL, 2×10⁶ cells/mL, 3×10⁶ cells/mL, 4×10⁶ cells/mL, 5×10⁶ cells/mL, 6×10⁶ cells/mL, 7×10⁶ cells/mL, 8×10⁶ cells/mL, 9×10⁶ cells/mL, or 1×10⁷ cells/mL). For example, the density of the hepatocytes may be, e.g., 3×10⁵ cells/mL.

In some embodiments, the plurality of cell populations further includes one or more (e.g., one, two, three, four, five, six, seven, eight, nine, ten, or more) additional cell populations.

In some embodiments, the bioreactor has a capacity of 0.1 L to 500 L, e.g., 0.1 L to 10 L (0.1 L, 0.2 L, 0.3 L, 0.4 L, 0.5 L, 0.6 L, 0.7 L, 0.8 L, 0.9 L, or 1 L), 1 L to 10 L (e.g., 1 L, 2 L, 3 L, 4 L, 5 L, 6 L, 7 L, 8 L, 9 L, or 10 L), 10 L to 100 L (e.g., 10 L, 20 L, 30 L, 40 L, 50 L, 60 L, 70 L, 80 L, 90 L, or 100 L), or 100 L to 500 L (e.g., 100 L, 150 L, 200 L, 250 L, 300 L, 350 L, 400 L, 450 L, or 500 L). In some embodiments, the bioreactor has a capacity of 100 L to 300 L. In some embodiments, the bioreactor has a capacity of 0.1 L to 1 L (e.g., 0.5 L).

In some embodiments, the first population of cells and the second population of cells do not expand (e.g., in cell number) by more than 30% during the agitating step. For example, in some embodiments, the first population of cells and the second population of cells do not expand by more than 230%, 25%, 20%, 15%, 10%, 9%, 8%, 7%, 6%, 5%, 4%, 3%, 2%, or 1%.

The bioreactor used in a method described herein may be a stir tank bioreactor (e.g., single use stir tank bioreactor) or a vertical wheel bioreactor. The bioreactor may further include a bioprocess controller that controls one or more of pH, temperature, and dissolved oxygen concentration.

In some embodiments, the first population of cells and the second population of cells do not adhere to the bioreactor.

In some embodiments, at least 80% (e.g., at least 85%, 90%, 95%, 97%, or 99%) of the aggregates have a mean diameter±10% (e.g., +10%, 9%, 8%, 7%, 6%, 5%, 4%, 3%, 2%, or 1%) of each other.

In some embodiments, at least 80% (e.g., at least 85%, 90%, 95%, 97%, or 99%) of the aggregates have a mean diameter of less than 200 μm. In some embodiments, at least 80% (e.g., at least 85%, 90%, 95%, 97%, or 99%) of the aggregates have an area/perimeter ratio of less than or equal to 45, e.g., less than or equal to 40, 35, 30, 25, 20, 15, or 10.

In some embodiments, the aggregates are spheroids.

In some embodiments, the agitating step has a duration of from 1 hour to 72 hours (e.g., 6 hours to 72 hours, 6 hours to 48 hours, 12 hours to 48 hours, or 12 hours to 24 hours). For example, in some embodiments, the agitating step includes a duration of up to 48 hours. In some embodiments, the agitating step includes a duration of up to 24 hours. In some embodiments, the agitating step includes a duration of up to 18 hours. In some embodiments, the agitating step includes a duration of up to 12 hours. In some embodiments, the agitating step includes a duration of 12 to 24 hours. In some embodiments, the agitating step includes a duration of 12 to 48 hours. In some embodiments, the agitating step includes a duration of 24 to 48 hours.

In some embodiments, the agitation includes rotating the bioreactor at a speed of 10 rotations per minute (RPM) to 50 RPM (e.g., 15 RPM, 20 RPM, 25 RPM, 30 RPM, 35 RPM, 40 RPM, 45 RPM, or 50 RPM, e.g., 36 RPM).

In some embodiments, the medium has a viscosity of 0.5 cP to 2 cP (e.g., 0.9 cP to 1.4 cP, e.g., 0.5 cP, 0.6 cP, 0.7 cP, 0.8 cP, 0.9 cP, 1 cP, 1.1 cP, 1.2 cP, 1.3 cP, 1.4 cP, 1.5 cP, 1.6 cP, 1.7 cP, 1.8 cP, 1.9 cP, or 2 cP).

In some embodiments, the medium includes 1-20 μg/ml (e.g., 1 μg/mL, 2 μg/mL, 3 μg/mL, 4 μg/mL, 5 μg/mL, 6 μg/mL, 7 μg/mL, 8 μg/mL, 9 μg/mL, 10 μg/mL, 11 μg/mL, 12 μg/mL, 13 μg/mL, 14 μg/mL, 15 μg/mL, 16 μg/mL, 17 μg/mL, 18 μg/mL, 19 μg/mL, or 20 μg/mL) recombinant human insulin, 1-10 μg/mL (e.g., 1 μg/mL, 2 μg/mL, 3 μg/mL, 4 μg/mL, 5 μg/mL, 6 μg/mL, 7 μg/mL, 8 μg/mL, 9 μg/mL, or 10 μg/mL) human transferrin, and 1×10⁻³ to 1×10⁻² μg/mL (e.g., 1×10⁻³ μg/mL, 2×10⁻³ μg/mL, 3×10⁻³ μg/mL, 4×10⁻³ μg/mL, 5×10⁻³ μg/mL, 6×10⁻³ μg/mL, 7×10⁻³ μg/mL, 8×10⁻³ μg/mL, 9×10⁻³ μg/mL, or 1×10⁻² μg/mL) selenite.

In some embodiments, the medium includes laminin, collagen, elastin, or fibronectin. For example, the medium may include 1-10 μg/mL (e.g., 1 μg/mL, 2 μg/mL, 3 μg/mL, 4 μg/mL, 5 μg/mL, 6 μg/mL, 7 μg/mL, 8 μg/mL, 9 μg/mL, or 10 μg/mL) laminin. In some embodiments, the medium includes fibrinogen.

In some embodiments, the medium includes 1-20 μM (e.g., 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, or 20 μM) Rho-associated protein kinase (ROCK) inhibitor. The ROCK inhibitor may be, e.g., Y27632.

In some embodiments, the medium includes human serum. For example, the medium may include from 0.1% to 20% (v/v) human serum, e.g., 0.1% to 1% (e.g., 0.1%, 0.2%, 0.3%, 0.4%, 0.5%, 0.6%, 0.7%, 0.8%, 0.9%, or 1%), e.g., 1% to 10% (e.g., 1%, 2%, 3%, 4%, 5%, 6%, 7%, 8%, 9%, or 10%), or 10% to 20% (e.g., 10%, 11%, 12%, 13%, 14%, 15%, 16%, 17%, 18%, 19%, or 20%). In some embodiments, the medium includes 1% to 10% (v/v) human serum. In some embodiments, the medium includes 10% (v/v) human serum.

In some embodiments, the medium includes platelet lysate (e.g., human platelet lysate, e.g., PLATELET GOLD™). For example, the medium may include 0.1% to 10% (v/v) platelet lysate, e.g., 0.1% to 1% (e.g., 0.1%, 0.2%, 0.3%, 0.4%, 0.5%, 0.6%, 0.7%, 0.8%, 0.9%, or 1%) or 1% to 10% (e.g., 2%, 3%, 4%, 5%, 6%, 7%, 8%, 9%, or 10%) human platelet lysate. In some embodiments, the medium includes 1% to 5% (v/v) platelet lysate.

In some embodiments, the medium includes glucose (e.g., from 4,000 mg/L to 5,000 mg/L glucose, e.g., 4,100 mg/L, 4,200 mg/L, 4,300 mg/L, 4,400 mg/L, 4,500 mg/L, 4,600 mg/L, 4,700 mg/L, 4,800 mg/L, 4,900 mg/L, or 5,000 mg/L glucose). In some embodiments, the medium includes 4,500 mg/L glucose.

In some embodiments, the medium includes glucagon (e.g., from 10 ng/ml to 100 ng/mL glucagon, e.g., 10 ng/ml, 20 ng/ml, 30 ng/ml, 40 ng/ml, 50 ng/mL, 60 ng/ml, 70 ng/ml, 80 ng/ml, 90 ng/mL, or 100 ng/ml glucagon). In some embodiments, the medium includes 40 ng/ml glucagon.

In some embodiments, the medium includes dexamethasone (e.g., from 10 ng/ml to 100 ng/ml dexamethasone, e.g., 10 ng/ml, 20 ng/mL, 30 ng/mL, 40 ng/mL, 50 ng/ml, 60 ng/ml, 70 ng/ml, 80 ng/ml, 90 ng/mL, or 100 ng/ml dexamethasone). In some embodiments, the medium includes 60 ng/ml dexamethasone.

In some embodiments, the bioreactor is incubated at temperature of 35° C. to 39° C. (e.g., 35° C., 35.5° C., 36° C., 36.5° C., 37° C., 37.5° C., 38° C., 38.5° C. or 39° C.).

In some embodiments, the method produces a density of aggregates of 500 aggregates/mL to 10,000 aggregates/mL (e.g., 500 aggregates/mL, 600 aggregates/mL, 700 aggregates/mL, 800 aggregates/mL, 900 aggregates/mL, 1,000 aggregates/mL, 2,000 aggregates/mL, 3,000 aggregates/mL, 4,000 aggregates/mL, 5,000 aggregates/mL, 6,000 aggregates/mL, 7,000 aggregates/mL, 8,000 aggregates/mL, 9,000 aggregates/mL, or 10,000 aggregates/mL).

In some embodiments, the method produces an average aggregate mean diameter of from 50 μm to 200 μm (e.g., 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, or 200μm).

In some embodiments, the method produces an average total volume of aggregates of 200 μL to 50 mL, e.g., from 200 μL to 1 mL (e.g., 200 μL, 250 μL, 300 μL, 350 μL, 400 μL, 450 μL, 500 μL, 550 μL, 600 μL, 650 μL, 700 μL, 750 μL, 800 μL, 850 μL, 900 μL, 950 μL, or 1 mL), from 1 mL to 10 ml (e.g., 2 mL, 3 mL, 4 mL, 5 mL, 6 mL, 7 mL, 8 mL, 9 mL, or 10 mL), or from 10 mL to 100 ml (e.g., 20 mL, 30 mL, 40 mL, 50 mL, 60 mL, 70 mL, 80 mL, 90 mL, or 100 mL).

In some embodiments, the method produces an average total mass of aggregates of 100 mg to 100 g, e.g., from 100 mg to 1 g (e.g., 100 mg, 200 mg, 300 mg, 400 mg, 500 mg, 600 mg, 700 mg, 800 mg, 900 mg, or 1 g), from 1 g to 10 g (e.g., 1 g, 2 g, 3 g, 4 g, 5 g, 6 g, 7 g, 8 g, 9 g, or 10 g), or 10 g to 100 g (e.g., 10 g, 20 g, 30 g, 40 g, 50 g, 60 g, 70 g, 80 g, 90 g, or 100 g).

In some embodiments, collecting the aggregates includes pipetting, pouring, decanting, or draining the bioreactor. In some embodiments, the bioreactor includes a collection tube, and the method includes collecting the aggregates in the collection tube.

In some embodiments, the method further includes the step of washing the collected aggregates of step (b).

In some embodiments, the method further includes the step of purifying the collected aggregates of step (b). The collected aggregates may be purified, e.g., by centrifugation or acoustic separation. For example, the collected aggregates may be purified by counter flow centripetal centrifugation or density gradient centrifugation.

In some embodiments, at least 80% (e.g., at least 80%, 85%, 90%, 95%, 97%, or 99%) of the aggregates have a mean diameter±10% (e.g., ±10%, 9%, 8%, 7%, 6%, 5%, 4%, 3%, 2%, or 1%) of each other after purification.

In some embodiments, the method further includes the step of concentrating the collected aggregates of step (b).

In some embodiments, the method further includes the step of formulating the collected aggregates of step (b) in a storage buffer.

In some embodiments, the biocompatible scaffold further includes a reinforcing agent. The reinforcing agent may include, for example, collagen, poly (ethylene glycol), polyvinylidene acetate

(PVDA), polyvinylidene fluoride (PVDF), poly (lactic-co-glycolic) acid (PLGA), or poly (I-lactic acid) (PLLA). In some embodiments, the aggregates (e.g., at least 80%, e.g., at least 85%, 90%, 95%, 97%, or 99% of the aggregates) each include at least 1×10³ cells (e.g., at least 1×10³ cells, 2×10³ cells, 3×10³ cells, 4×10³ cells, 5×10³ cells, 6×10³ cells, 7×10³ cells, 8×10³ cells, 9×10³ cells, or 1×10⁴ cells). In some embodiments, the aggregates each include at least 1×10⁵ cells, 1×10⁶ cells, 1×10⁷ cells, or 1×10⁸ cells.

In some embodiments, the aggregates (e.g., at least 80%, e.g., at least 85%, 90%, 95%, 97%, or 99% of the aggregates) each have a mean diameter of at least 50 μm (e.g., at least 50 μm, 60 μm, 70 μm, 80 μm, 90 μm, 100 μm, or more.

In some embodiments, at least 80% (e.g., at least 85%, 90%, 95%, 97%, or 99%) of the aggregates have a mean diameter of less than 200 μm. In some embodiments, at least 80% (e.g., at least 85%, 90%, 95%, 97%, or 99%) of the aggregates have an area/perimeter ratio of less than or equal to 45, e.g., less than or equal to 40, 35, 30, 25, 20, 15, or 10.

In some embodiments, the method further includes washing the cells prior to step (a), e.g., after thawing the cells.

In some embodiments, the method further includes the step of (c) encapsulating the collected aggregates of step (b) in a biocompatible scaffold.

In another aspect, the invention features a method of encapsulating aggregates of a plurality of cell populations that includes a first population of cells and a second population of cells. The method includes the step of (a) agitating a liquid medium that contains the first population of cells and the second population of cells in a bioreactor for a duration sufficient to form aggregates that include the first population of cells and the second population of cells. The first population of cells and the second population of cells are in suspension in the liquid medium during the agitating step. The method produces aggregates of the first population of cells and the second population of cells. The further includes the step of (b) collecting the aggregates of step (a), e.g., that include the first population of cells and the second population of cells. The method also includes the step of (c) encapsulating the aggregates of step (b) in a biocompatible scaffold.

In some embodiments, encapsulating the aggregates includes providing a polymerizing agent or a cross-linking reagent to polymerize or cross-link the biocompatible scaffold, thereby encapsulating the aggregates.

The biocompatible scaffold may include, for example, fibrinogen. The polymerizing agent may include, for example, thrombin. In some embodiments, the thrombin polymerizes the fibrinogen into fibrin.

In some embodiments, the method further includes the step of washing the collected aggregates of step (b) prior to encapsulation.

In some embodiments, the method further includes washing the cells prior to step (a), e.g., after thawing the cells.

In some embodiments, the method further includes the step of purifying the collected aggregates of step (b) prior to encapsulation. The collected aggregates may be purified, e.g., by centrifugation or acoustic separation. For example, the collected aggregates may be purified by counter flow centripetal centrifugation or density gradient centrifugation.

In some embodiments, at least 80% (e.g., at least 80%, 85%, 90%, 95%, 97%, or 99%) of the aggregates have a mean diameter±10% (e.g., ±10%, 9%, 8%, 7%, 6%, 5%, 4%, 3%, 2%, or 1%) of each other after purification.

In some embodiments, the method further includes the step of concentrating the collected aggregates of step (b) prior to encapsulation.

In some embodiments, the method further includes the step of formulating the collected aggregates of step (b) in a storage buffer prior to encapsulation.

### Definitions

As used herein, the term “agitating” refers to providing movement of a liquid medium in a cell culture such that the contents of the cell culture remain in suspension and do not settle or adhere to a bioreactor. Agitation can be accomplished by any suitable method known in the art, such as stirring, shaking, rotating, spinning, or the like.

As used in the context of the present disclosure, an “engineered tissue construct” refers to a mixture of cultured cells (e.g., parenchymal cells (e.g., hepatocytes (e.g., primary human hepatocytes))) and, optionally, stromal cells (e.g., fibroblasts e.g., neonatal foreskin fibroblasts), and a biocompatible scaffold (e.g., a biocompatible hydrogel scaffold, e.g., fibrin). The relative volume of the engineered tissue construct may be between 0.1 mL to 5 L.

Cells can be from established cell lines, or they can be primary cells, where “primary cells,” “primary cell lines,” and “primary cultures” are used interchangeably herein to refer to cells and cells cultures that have been derived from and allowed to grow in vitro for a limited number of passages, e.g., splitting, of the culture. For example, primary cultures can be cultures that have been passaged 0 times, 1 time, 2 times, 4 times, 5 times, 10 times, or 15 times, but not enough times go through the crisis stage. Primary cell lines can be maintained for fewer than 10 passages in vitro. If the cells are primary cells, such cells can be harvested from an individual by any convenient method. For example, cells from tissues such as skin, muscle, bone marrow, spleen, liver, pancreas, lung, intestine, stomach, etc. are conveniently harvested by biopsy. An appropriate solution can be used for dispersion or suspension of the harvested cells. Such solution will generally be a balanced salt solution, e.g., normal saline, phosphate-buffered saline (PBS), Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc. The cells can be used immediately, or they can be stored, frozen, for long periods of time, being thawed and capable of being reused. In such cases, the cells will usually be frozen in 10% DMSO, 50% serum, 40% buffered medium, or some other such solution as is commonly used in the art to preserve cells at such freezing temperatures and thawed in a manner as commonly known in the art for thawing frozen cultured cells. For example, hepatocytes may be isolated by conventional methods (Berry and Friend, 1969, J. Cell Biol. 43:506-520) which can be adapted for human liver biopsy or autopsy material (e.g., to garner primary human hepatocytes).

As used herein, the term “cell type” refers to a group of cells sharing a phenotype that is statistically separable based on gene expression data. For example, cells of a common cell type may share similar structural and/or functional characteristics, such as similar gene activation patterns and antigen presentation profiles. Cells of a common cell type may include those that are isolated from a common tissue (e.g., epithelial tissue, neural tissue, connective tissue, or muscle tissue) and/or those that are isolated from a common organ, tissue system, blood vessel, or other structure and/or region in an organism.

As used herein, a scaffold (e.g., a hydrogel scaffold) is considered “biocompatible” when is it does not exhibit toxicity when introduced into a subject (e.g., a human). In the context of the present disclosure, it is preferable that the biocompatible scaffold does not exhibit toxicity towards the cells of an engineered tissue construct or when implanted in vivo in a subject (e.g., a human). For example, with respect to hepatocytes, hepatotoxicity can be measured, for example, by determining hepatocytes apoptotic death rate (e.g., wherein an increase in apoptosis is indicative of hepatotoxicity), transaminase levels (e.g., wherein an increase in transaminase levels is indicative of hepatotoxicity), ballooning of the hepatocytes (e.g., wherein an increase in ballooning is indicative of hepatotoxicity), microvesicular steatosis in the hepatocytes (e.g., wherein an increase in steatosis is indicative of hepatotoxicity), biliary cells death rate (e.g., wherein an increase in biliary cells death rate is indicative of hepatotoxicity), Y-glutamyl transpeptidase (GGT) levels (e.g., wherein an increase in GGT levels is indicative of hepatotoxicity). A biocompatible scaffold can include, but is not limited to, fibrin and heparin. The biocompatible scaffold maybe a biocompatible hydrogel scaffold.

As used herein, the term “hydrogel” refers to a network of polymer chains that are hydrophilic in nature, such that the material absorbs a high volume of water or other aqueous solution. Hydrogels can include, for example, at least 70% v/v water, at least 80% v/v water, at least 90% v/v water, at least 95%, 96%, 97%, 98% and even 99% or greater v/v water (or other aqueous solution). Hydrogels can include natural or synthetic polymers, the polymeric network often featuring a high degree of crosslinking. Hydrogels also possess a degree of flexibility very similar to natural tissue, due to their significant water content. Hydrogels are particularly useful in tissue engineering applications as scaffolds for culturing cells. In certain embodiments, the hydrogels are made of biocompatible polymers.

### BRIEF DESCRIPTION OF THE DRAWINGS

FIGS. 1A and 1B are graphs showing cell count per mL as a function of time when aggregated under suspension conditions in ITS (insulin-transferrin-selenium) media alone, or with laminin or ROCK inhibitor. FIG. 1A shows live cells, and FIG. 1B shows dead cells.

FIGS. 2A-2D are graphs showing aggregate data in different culture conditions. FIG. 2A shows seed (i.e., aggregate) count. FIG. 2B shows aggregate mean diameter. FIG. 2C shows debris count.

FIG. 2D shows superaggregate (SuperAgg) count. μwell baseline=comparison condition in which cells were aggregated in microwells.

FIG. 3 is a series of graphs showing aggregate (seed) diameter distributions in different culture conditions.

FIG. 4 is a set of photomicrographs showing superaggregates formed under different culture conditions.

FIGS. 5A-5D are graphs showing aggregate data in different culture conditions at 24 or 48 hours of incubation. FIG. 5A shows seed/aggregate count. FIG. 5B shows aggregate mean diameter. FIG. 5C shows debris count. FIG. 5D shows superaggregate count.

FIG. 6 is a set of photomicrographs showing aggregates formed under different culture conditions.

FIGS. 7A-7D are graphs showing aggregate (seed) data with ITS only or in the presence of ROCK inhibitor. FIG. 7A shows aggregate (seed) size in different replicates. FIG. 7B shows aggregate (seed) concentration in different replicates. FIG. 7C shows aggregate (seed) size under different replicates. FIG. 7D shows debris concentration under different replicates.

FIGS. 8A and 8B are graphs showing ammonia clearance. FIG. 8A shows ammonia clearance. FIG. 8B shows the same data with the acellular background subtracted.

FIGS. 9A and 9B are graphs showing ammonia clearance in aggregates formed under suspension conditions as compared to aggregates formed in microwells. FIG. 9A shows ammonia clearance relative to a comparison condition with microwells and FIG. 9B shows ammonia clearance relative to a comparison condition with microwells at 18 hours and 42 hours.

FIG. 10 is a table showing aggregate (seed) characteristics under different culture conditions with ITS only or in the present of ROCK inhibitor.

FIG. 11 is an exemplary set of photomicrographs and graphs showing aggregate (seed) size of aggregates formed in ITS. Seed count: 2052; seed concentration (assuming 300 μL): 6840.0 seeds/mL; seed mean diameter: 86.8 μm; seed median diameter: 81.6 μm; debris score: 11321; supper aggregate count: 2.

FIG. 12 is an exemplary set of photomicrographs and graphs showing aggregate (seed) size of aggregates formed in the presence of ROCK inhibitor. Seed count: 1160; seed concentration (assuming 300 μL): 3866.7 seeds/mL; seed mean diameter: 102.5 μm; seed median diameter: 97.3 μm; debris score: 2045; supper aggregate count: 1.

FIGS. 13A and 13B are graphs showing cell concentrations over 24 hours of incubation under suspension conditions. FIG. 13A shows live cells, and FIG. 13B shows dead cells.

FIGS. 14A-14D are graphs showing aggregate data at various concentrations of human serum (HS) and platelet gold (PG; human lysate). FIG. 14A shows aggregate (seed) size. FIG. 14B shows average aggregate (seed) volume. FIG. 14C shows aggregate (seed) concentration. FIG. 14D shows aggregate (seed) total volume.

FIGS. 15A-15D are graphs showing aggregate data produced under various culture conditions. FIG. 15A shows aggregate (seed) size. FIG. 15B shows average aggregate (seed) volume. FIG. 15C shows average aggregate (seed) volume. FIG. 15D shows total aggregate (seed) volume. VWB_005 and VWB_006=ITS, fibrinogen, ROCK inhibitor, laminin, and 10% fetal bovine serum; VWB_007=human serum and platelet lysate.

FIG. 16 is a graph showing lactate dehydrogenase (LDH) formed at 24 hours under different culture conditions, including ITS, platelet gold, and human serum conditions.

FIG. 17 is a set of photomicrographs showing overlaid live and dead fluorescent staining channels on phase-contrast images of aggregates harvested at 24 hours.

FIG. 18 is a graph showing the number of viable and dead cells (normal human dermal fibroblasts) after washing when thawed or washed with a CTS™ ROTEA™ harvest, which uses a counter flow centrifugation system. Viability was unchanged from 98% to 97%. Recovery was ˜87%.

FIGS. 19A-19C are graphs showing the number of viable and dead cells (primary human hepatocytes (PHH)) after washing using a CTS™ ROTEA™ counter flow centrifugation system at different flow rates of wash media. FIG. 19A shows 30 mL/min, FIG. 19B shows 60 mL/min, and FIG. 19C shows 17 mL/min.

FIGS. 20A and 20B are graphs showing viable cells after washing using a CTS™ ROTEA™ counter flow centrifugation system. FIG. 20A shows % viable cells as a function of flow rate. FIG. 20B shows the % change in dead cell number as a function of flow rate.

FIG. 21 is a set of graphs showing aggregate (seed) data before (left) and after (right) washing of microwell based aggregates using a CTS™ ROTEA™ counter flow centrifugation system.

FIG. 22 is a graph showing % recovery as a function of aggregate size.

FIG. 23 is a set of graphs showing aggregate (seed) data before (top) and after (bottom) washing and concentration of vertical wheel bioreactor-based aggregates using a CTS™ ROTEA™ counter flow centrifugation system.

FIG. 24 is a graph showing estimate harvest (%) as a function of aggregate size in two replicates.

FIGS. 25A-25D are a set of graphs showing seed count (FIG. 25A), seed concentration (FIG. 25B), seed diameter (FIG. 25C), and debris count (FIG. 25D) for the indicated conditions (ITS, 10% human serum, 5% human serum, or 5% Platelet Gold) after 24 hours of aggregation.

FIG. 26 is a graph showing super-aggregate count for the indicated media conditions after 24 hours of suspension-induced aggregation.

FIGS. 27A and 27B are graphs showing average seed volume and seed volume per mL of culture for the indicated media conditions.

FIGS. 28A and 28B are graphs showing normalized values of LDH (mU) per reactor (aggregation) (FIG. 28A) or per plate (encapsulation) (FIG. 28B). HS, human serum; PG; PLATELET GOLD™ human platelet lysate.

FIGS. 29A and 29B are graphs showing alanine transaminase (ALT) (FIG. 29A) and aspartate transaminase (AST) (FIG. 29B) (U/L) released by PHH and NHDF cell suspension during 24 hours of VWB aggregation. n=3 samples from each reactor condition. Mean and 95% CI are plotted. Values plotted account for initial level of ALT and AST at 0 hours (aggregation initiation).

FIGS. 30A and 30B are graphs showing ammonia clearance. FIG. 30A shows normalized ammonia concentrations including the acellular control. FIG. 30B shows experimental group relative ammonia with acellular controls subtracted/removed. n=4 per condition, mean and 95% CI plotted. Dashed line is at 1.0 relative ammonia (100% relative ammonia).

FIG. 31 is a graph showing relative level of cleared ammonia at 72 hours. n=4 grafts per condition. Mean and 95% CI plotted.

FIG. 32 is a graph showing ammonia clearance k-values of grafts made from the different aggregation media formulations tested. n=4 grafts per condition, mean is plotted. Constraints: k>0.

FIG. 33 is a graph showing seed concentration as a function of aggregation time for the indicated experimental conditions.

FIG. 34 is a graph showing seed diameter as a function of aggregation time for the indicated experimental conditions.

FIGS. 35A and 35B are graphs showing the total number of residual cells (FIG. 35A) or dead cells (FIG. 35B) as a function of aggregation time for the indicated experimental conditions.

FIGS. 36A and 36B are graphs showing AST secreted during aggregation as a function of aggregation time (FIG. 36A) and AST normalized by PHH concentration as a function of aggregation time (FIG. 36B) for the indicated experimental conditions.

FIG. 37 is a graph showing seed viability as a function of aggregation time for the indicated experimental conditions.

FIGS. 38A and 38B are a set of graphs showing U1 potency screen of ammonia clearance with control present (FIG. 38A) or subtracted (FIG. 38B).

FIGS. 39A and 39B are graphs showing U1-surroseed total signal (human albumin) with FIBRYGA® control present (FIG. 39A) or subtracted (FIG. 39B). Groups: 1: microwell, Lonza, RUO Fibrin; 2: VWB, ABM, FIBRYGAR; 3: microwell, ABM, FIBRYGA®; 4: microwell, ABM, RUO Fibrin; 5: VWB, ABM, RUO Fibrin; 6: microwell, Lonza, FIBRYGAR; 7: GMP FIBRYGA® (Acellular control).

FIG. 40 is a schematic drawing showing a timeline of graft implantation in mice.

FIGS. 41A-41C are graphs showing in vivo plasma biomarker trends. FIG. 41A shows albumin, FIG. 41B shows transferrin, and FIG. 41C shows a correlation between albumin and transferrin. Groups:

1: Baseline (microwell, Lonza expanded, RUO Fibrin); 2: New (VWB, ABM T&A, FIBRYGA®); 3: Control (microwell, ABM, ABM T&A, GMP Fibrin); 4: Control (microwell, ABM T&A, RUO Fibrin; 5: Control (VWB, ABM T&A, RUO Fibrin); 6: Control (acellular GMP fibrin).

FIG. 42 is an image showing an example CK18 stained hepatocyte image.

FIG. 43 is an image showing an example CD31 stained vessel image.

FIG. 44 is an image showing an example of detection of hepatocyte aggregates with cell profiler.

FIG. 45 is an image showing an example of expanded regions around hepatocytes to define region of interest for vascular analysis.

FIG. 46 is an image showing an example of mask resultant from ROI definition based on hepatocyte locations.

FIG. 47 is an image showing an example of CD31 vessel region down selected by mask.

FIG. 48 is a graph showing average vessel length. Data are mean with 95% confidence interval.

FIG. 49 is a graph showing number of hepatocyte aggregates per mm².

FIG. 50 is graph showing number of vessels per mm².

FIG. 51 is a graph showing number of vessels per number of hepatocyte aggregates.

FIG. 52 is a graph showing a correlation evaluation between secreted human albumin levels and aggregate density.

FIG. 53 is a graph showing a correlation evaluation between vessels per aggregate and secreted albumin.

FIG. 54 is a graph showing seed layer height as a function of dose density of seeds produced by a vertical wheel bioreactor with and without washing with ROTEA™ counterflow centrifugation system.

FIG. 55 is a set of images showing graft cross-sections of seed layer heights of various doses and seed types; VWB=vertical wheel bioreactor.

FIGS. 56A and 56B are graphs showing corrected ALT (FIG. 56A) and AST (FIG. 56B) levels in aggregating cells in volumes of 60 mL, 75 mL, 100 mL, and 375 mL at 0 and 22 hours.

FIG. 57 is a graph showing partial oxygen pressure in aggregating cells in volumes of 60 mL, 75 mL, 100 mL, and 375 mL up to 22 hours.

FIGS. 58A and 58B are graphs showing lactate dehydrogenase (LDH) concentrations (mU) at 0 hours, 24 hours, and 60 hours from normal human dermal fibroblasts (NHDF), primary human hepatocytes (PHH), and a combination of NHDF and PHH. FIG. 58A shows extracellular LDH, and FIG. 58B shows total LDH.

### DETAILED DESCRIPTION

Formation of cell aggregates for tissue engineering is critical as a first step towards creating useful tissues for various therapeutic approaches. However, mimicking biological conditions to achieve such conditions remains challenging. In particular, many methods utilize two dimensional approaches, which may only create flat layers of tissue, as compared to the three-dimensional structures required for many tissues. Moreover, employing facile manipulation of cell aggregates remains challenging due to the requirement for various cell media exchanges to ensure robust cell growth and aggregation.

The present invention solves this problem by providing a bioreactor-based method of producing aggregates in suspension in a liquid medium. The method includes providing a plurality of cell populations that includes a first population of cells and a second population of cells and agitating a liquid medium with the cell populations in the bioreactor for a duration sufficient to form aggregates of the first and second populations of cells. The present invention is based in part on the surprising discovery that aggregates of two different populations of cells can form in an agitated liquid medium in a bioreactor despite maintaining the cells in suspension in which cell-to-cell contact, which is required for aggregation, was assumed to be minimal. Moreover, the methods reduce, eliminate, or minimize expansion while promoting aggregation, thus creating useful cell aggregates for various downstream applications, such as encapsulation into a biocompatible scaffold for tissue and organ engineering. Finally, using a suspension-based bioreactor allows scaling for production of commercially relevant quantities of cell aggregates.

### Methods of Producing Aggregates

The invention features a method of producing aggregates of a plurality of cell populations (e.g., a first population of cells and a second population of cells). The method includes the step of agitating a liquid medium that contains the first population of cells and the second population of cells in a bioreactor. The first population of cells and the second population of cells are in suspension in the liquid medium during the agitating step. The cells are agitated for a duration sufficient to form aggregates that include the first population of cells and the second population of cells. The method may further include collecting the aggregates of the first population of cells and the second population of cells. These aggregates may be used for subsequent downstream applications, such as encapsulation in a biocompatible scaffold to form an engineered tissue construct. Prior to encapsulation, the aggregates may be collected, washed, purified, concentrated, and/or formulated in a suitable storage medium (e.g., buffer).

In some embodiments, the first population of cells and the second population of cells do not expand (e.g., in cell number) by more than 30% during the agitating step. For example, in some embodiments, the first population of cells and the second population of cells do not expand by more than 30%, 25%, 20%, 15%, 10%, 9%, 8%, 7%, 6%, 5%, 4%, 3%, 2%, or 1%.

In some embodiments, the first population of cells and the second population of cells do not adhere to the bioreactor.

In some embodiments, the agitating step has a duration of from 1 hour to 72 hours (e.g., 6 hours to 72 hours, 6 hours to 48 hours, 12 hours to 48 hours, or 12 hours to 24 hours, e.g., 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 49 hours, 50 hours, 51 hours, 52 hours, 53 hours, 54 hours, 55 hours, 56 hours, 57 hours, 58 hours, 59 hours, 60 hours, 61 hours, 62 hours, 63 hours, 64 hours, 65 hours, 66 hours, 67 hours, 68 hours, 69 hours, 70 hours, 71 hours, or 72 hours). For example, in some embodiments, the agitating step includes a duration of up to 48 hours. In some embodiments, the agitating step includes a duration of up to 24 hours. In some embodiments, the agitating step includes a duration of up to 18 hours. In some embodiments, the agitating step includes a duration of up to 12 hours. In some embodiments, the agitating step includes a duration of 12 to 24 hours (e.g., 12 hours, 13 hours, 14 hours, 15 hours, 16 hours, 17 hours, 18 hours, 19 hours, 20 hours, 21 hours, 22 hours, 23 hours, or 24 hours). In some embodiments, the agitating step includes a duration of 12 to 48 hours (e.g., 12 hours, 13 hours, 14 hours, 15 hours, 16 hours, 17 hours, 18 hours, 19 hours, 20 hours, 21 hours, 22 hours, 23 hours, 24 hours, 30 hours, 36 hours, 42 hours, or 48 hours). In some embodiments, the agitating step includes a duration of 24 to 48 hours.

In some embodiments, the agitating step has a duration that is less than or equal to a doubling time of the first population of cells and/or the second population of cells. In some embodiments, the agitating step has a duration that is not more than 3-fold (e.g., no more than 2-fold or 1-fold) than the doubling time of the first population of cells and/or the second population of cells. Such a constraint may preclude an increase in the number of cells in order to promote aggregation without promoting expansion. For example, if a population of cells has a doubling time of 24-36 hours, in some embodiments the agitating step may not be more than 72 hours (i.e., no more than 2-fold greater than 24-hour doubling time).

In some embodiments, agitation can be accomplished by any suitable method known in the art, such as stirring, shaking, rotating, spinning, or the like.

In some embodiments, the agitation includes rotating the bioreactor at a speed of 10 RPM to 50 RPM (e.g., 15 RPM, 20 RPM, 25 RPM, 30 RPM, 35 RPM, 40 RPM, 45 RPM, or 50 RPM, e.g., 36 RPM).

In some embodiments, the bioreactor is incubated at temperature of 35° C. to 39° C. (e.g., 35° C., 35.5° C., 36° C., 36.5° C., 37° C., 37.5° C., 38° C., 38.5° C. or 39° C.).

In some embodiments, at least 80% (e.g., at least 85%, 90%, 95%, 97%, or 99%) of the aggregates have a mean diameter±10% (e.g., ±10%, 9%, 8%, 7%, 6%, 5%, 4%, 3%, 2%, or 1%) of each other. In some embodiments, the aggregates are spheroids.

One of skill in the art would appreciate that a population of aggregates may have a range of Z-average mean particle diameters within the population. Thus, the population may be polydisperse. The population may have a polydispersity index of 0.7 or less (e.g., 0.05 to 0.7, e.g., 0.05, 0.1, 0.15, 0.2, 0.25, 0.3, 0.35, 0.4, 0.45, 0.5, 0.6, 0.65, or 0.7). The polydispersity index can be determined using DLS (see, e.g., ISO 22412:2017). Purification may be used to reduce the polydispersity of the population of aggregates produced by the method, e.g., to produce a more monodisperse population.

In some embodiments, the method produces a density of aggregates of 500 aggregates/mL to 10,000 aggregates/mL (e.g., 500 aggregates/mL, 600 aggregates/mL, 700 aggregates/mL, 800 aggregates/mL, 900 aggregates/mL, 1,000 aggregates/mL, 2,000 aggregates/mL, 3,000 aggregates/mL, 4,000 aggregates/mL, 5,000 aggregates/mL, 6,000 aggregates/mL, 7,000 aggregates/mL, 8,000 aggregates/mL, 9,000 aggregates/mL, or 10,000 aggregates/mL).

In some embodiments, the method produces an average aggregate mean diameter of from 50 μm to 200 μm (e.g., 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, or 200 μm). In some embodiments, the aggregates (e.g., at least 80%, e.g., at least 85%, 90%, 95%, 97%, or 99% of the aggregates) each have a mean diameter of at least 50 μm (e.g., at least 50 μm, 60 μm, 70 μm, 80 μm, 90 μm, 100 μm, or more.

In some embodiments, at least 80% (e.g., at least 85%, 90%, 95%, 97%, or 99%) of the aggregates have a mean diameter of less than 200 μm. In some embodiments, at least 80% (e.g., at least 85%, 90%, 95%, 97%, or 99%) of the aggregates have an area/perimeter ratio of less than or equal to 45, e.g., less than or equal to 40, 35, 30, 25, 20, 15, or 10.

In some embodiments, the aggregates (e.g., at least 80%, e.g., at least 85%, 90%, 95%, 97%, or 99% of the aggregates) each include at least 1×10³ cells (e.g., at least 1×10³ cells, 2×10³ cells, 3×10³ cells, 4×10³ cells, 5×10³ cells, 6×10³ cells, 7×10³ cells, 8×10³ cells, 9×10³ cells, or 1×10⁴ cells). In some embodiments, the aggregates each include at least 1×10⁵ cells, 1×10⁶ cells, 1×10⁷ cells, or 1×10⁸ cells.

In some embodiments, the method produces an average total volume of aggregates of 200 μL to 50 mL, e.g., from 200 μL to 1 mL (e.g., 200 μL, 250 μL, 300 μL, 350 μL, 400 μL, 450 μL, 500 μL, 550 UL, 600 μL, 650 μL, 700 μL, 750 μL, 800 μL, 850 μL, 900 μL, 950 μL, or 1 mL), from 1 mL to 10 mL (e.g., 1 mL, 2 mL, 3 mL, 4 mL, 5 mL, 6 mL, 7 mL, 8 mL, 9 mL, or 10 mL), or from 10 mL to 100 ml (e.g., 10 mL, 20 mL, 30 mL, 40 mL, 50 mL, 60 mL, 70 mL, 80 mL, 90 mL, or 100 mL).

In some embodiments, the method produces an average total mass of aggregates of 100 mg to 100 g, e.g., from 100 mg to 1 g (e.g., 100 mg, 200 mg, 300 mg, 400 mg, 500 mg, 600 mg, 700 mg, 800 mg, 900 mg, or 1 g), from 1 g to 10 g (e.g., 1 g, 2 g, 3 g, 4 g, 5 g, 6 g, 7 g, 8 g, 9 g, or 10 g), or 10 g to 100 g (e.g., 10 g, 20 g, 30 g, 40 g, 50 g, 60 g, 70 g, 80 g, 90 g, or 100 g).

The methods described herein may further include collecting the aggregates from the bioreactor. Collecting the aggregates may include, for example, pipetting, pouring, decanting, or draining the bioreactor. In some embodiments, the bioreactor includes a collection tube, and the method includes collecting the aggregates in the collection tube.

In some embodiments, the method further includes the step of washing the collected aggregates, e.g., with a wash buffer, e.g., to remove any medium. Washing may include, for example, pipetting, pouring, decanting, or draining the medium. Washing may include dialyzing or exchanging the medium. The aggregates may be washed by centrifugation or acoustic separation. For example, the collected aggregates may be washed by counter flow centripetal centrifugation or density gradient centrifugation. The aggregates may be washed by a CTS™ ROTEA™ counter flow centrifugation system.

In some embodiments, the method further includes washing the cells prior to providing the cells in suspension culture, e.g., after thawing the cells.

In some embodiments, the method further includes the step of purifying the collected aggregates. The collected aggregates may be purified, e.g., by centrifugation or acoustic separation. For example, the collected aggregates may be purified by counter flow centripetal centrifugation or density gradient centrifugation. In some embodiments, at least 80% (e.g., at least 80%, 85%, 90%, 95%, 97%, or 99%) of the aggregates have a mean diameter±10% (e.g., ±10%, 9%, 8%, 7%, 6%, 5%, 4%, 3%, 2%, or 1%) of each other after purification.

In some embodiments, at least 80% (e.g., at least 85%, 90%, 95%, 97%, or 99%) of the aggregates have a mean diameter of less than 200 μm. In some embodiments, at least 80% (e.g., at least 85%, 90%, 95%, 97%, or 99%) of the aggregates have an area/perimeter ratio of less than or equal to 45, e.g., less than or equal to 40, 35, 30, 25, 20, 15, or 10.

The aggregates may be purified by a CTS ROTEA™ counter flow centrifugation system.

In some embodiments, the method further includes the step of concentrating the collected aggregates, e.g., to a concentration suitable for incorporation into a biocompatible scaffold.

In some embodiments, the method further includes the step of formulating the collected aggregates in a storage buffer, e.g., for storage or downstream use.

### Bioreactors

The methods described herein include agitating a plurality of cell populations in a bioreactor. The bioreactor may be any suitable bioreactor sufficient to agitate the cell populations to maintain the cells in suspension during agitation.

In some embodiments, the bioreactor has a capacity of 0.1 L to 500 L, e.g., 0.1 L to 10 L (0.1 L, 0.2 L, 0.3 L, 0.4 L, 0.5 L, 0.6 L, 0.7 L, 0.8 L, 0.9 L, or 1 L), 1 L to 10 L (e.g., 1 L, 2 L, 3 L, 4 L, 5 L, 6 L, 7 L, 8 L, 9 L, or 10 L), 10 L to 100 L (e.g., 10 L, 20 L, 30 L, 40 L, 50 L, 60 L, 70 L, 80 L, 90 L, or 100 L), or 100 L to 500 L (e.g., 100 L, 150 L, 200 L, 250 L, 300 L, 350 L, 400 L, 450 L, or 500 L). In some embodiments, the bioreactor has a capacity of 100 L to 300 L. In some embodiments, the bioreactor has a capacity of 0.5 L.

The bioreactor used in a method described herein may be a stir tank bioreactor (e.g., single use stir tank bioreactor) or a vertical wheel bioreactor. In some embodiments, the bioreactor is a BIOBLUE® c Single-Use Bioreactor (Eppendorf). In some embodiments, the bioreactor is a SCIVARIO® twin bioreactor (Eppendorf).

The bioreactor may further include a bioprocess controller. The bioprocess controller may control one or more of pH, temperature, and dissolved oxygen concentration.

In some embodiments, the first population of cells and the second population of cells do not adhere to the bioreactor. Accordingly, the bioreactor may include a coating to prevent adherence by the cells.

In some embodiments, the bioreactor (e.g., vertical wheel bioreactor) rotates at a speed of 10 RPM to 50 RPM (e.g., 15 RPM, 20 RPM, 25 RPM, 30 RPM, 35 RPM, 40 RPM, 45 RPM, or 50 RPM, e.g., 36 RPM).

In some embodiments, the bioreactor includes a collection tube, and the method includes collecting the aggregates in the collection tube.

### Aggregation Media

The methods described herein include agitating cells populations in a liquid medium to form aggregates. The liquid medium may be any suitable medium sufficient to form aggregates. The medium may include, for example, serum (e.g., human serum) or serum replacement.

In some embodiments, the medium has a viscosity of 0.5 cP to 2 cP (e.g., 0.9 cP to 1.4 cP, e.g., 0.5 cP, 0.6 cP, 0.7 cP, 0.8 cP, 0.9 cP, 1 cP, 1.1 cP, 1.2 cP, 1.3 cP, 1.4 cP, 1.5 cP, 1.6 cP, 1.7 cP, 1.8 cP, 1.9 cP, or 2 cP). The viscosity may be tuned to optimize cell-to-cell contact to enhance aggregation.

In some embodiments, the medium includes recombinant 1-20 μg/mL recombinant human insulin, 1-10 μg/mL human transferrin, and 1×10⁻³ to 1×10⁻² μg/mL selenite.

In some embodiments, the medium includes laminin, collagen, elastin, or fibronectin. For example, the medium may include 1-10 μg/mL (e.g., 1 μg/mL, 2 μg/mL, 3 μg/mL, 4 μg/mL, 5 μg/mL, 6 μg/mL, 7 μg/mL, 8 μg/mL, 9 μg/mL, or 10 μg/mL) laminin.

In some embodiments, the medium includes 1-20 μM (e.g., 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, or 20 μM) Rho-associated protein kinase (ROCK) inhibitor. The ROCK inhibitor may be, e.g., Y27632. Other ROCK inhibitors include, but are not limited to, AT-13148, BA-210, β-elemene, chroman 1, DJ4, fasudil, GSK-576371, GSK429286A, H-1152, hydroxyfasudil, LX-7101, netarsudil, RKI-1447, ripasudil, TCS-7001, thiazovivin, verosudil, Y-30141, Y33075, and Y-39983.

In some embodiments, the medium includes human serum. For example, the medium may include from 0.1% to 20% (v/v) human serum, e.g., 0.1% to 1% (e.g., 0.1%, 0.2%, 0.3%, 0.4%, 0.5%, 0.6%, 0.7%, 0.8%, 0.9%, or 1%), e.g., 1% to 10% (e.g., 1%, 2%, 3%, 4%, 5%, 6%, 7%, 8%, 9%, or 10%), or 10% to 20% (e.g., 10%, 11%, 12%, 13%, 14%, 15%, 16%, 17%, 18%, 19%, or 20%). In some embodiments, the medium includes 1% to 10% (v/v) human serum. In some embodiments, the medium includes 5% (v/v) human serum. In other embodiments, the medium includes 10% (v/v) human serum.

In some embodiments, the medium includes platelet lysate (e.g., human platelet lysate, e.g., PLATELET GOLD™). For example, the medium may include 0.1% to 10% (v/v) platelet lysate, e.g., 0.1% to 1% (e.g., 0.1%, 0.2%, 0.3%, 0.4%, 0.5%, 0.6%, 0.7%, 0.8%, 0.9%, or 1%) or 1% to 10% (e.g., 2%, 3%, 4%, 5%, 6%, 7%, 8%, 9%, or 10%) platelet lysate. In some embodiments, the medium includes 1% to 5% (v/v) platelet lysate. In some embodiments, the medium includes 5% (v/v) platelet lysate.

In some embodiments, the medium includes glucose (e.g., from 4,000 mg/L to 5,000 mg/L glucose, e.g., 4,100 mg/L, 4,200 mg/L, 4,300 mg/L, 4,400 mg/L, 4,500 mg/L, 4,600 mg/L, 4,700 mg/L, 4,800 mg/L, 4,900 mg/L, or 5,000 mg/L glucose). In some embodiments, the medium includes 4,500 mg/L glucose.

In some embodiments, the medium includes glucagon (e.g., from 10 ng/ml to 100 ng/mL glucagon, e.g., 10 ng/ml, 20 ng/ml, 30 ng/ml, 40 ng/ml, 50 ng/ml, 60 ng/mL, 70 ng/ml, 80 ng/ml, 90 ng/ml, or 100 ng/ml glucagon). In some embodiments, the medium includes 40 ng/ml glucagon.

In some embodiments, the medium includes fibrinogen.

In some embodiments, the medium includes dexamethasone (e.g., from 10 ng/ml to 100 ng/ml dexamethasone, e.g., 10 ng/ml, 20 ng/mL, 30 ng/ml, 40 ng/ml, 50 ng/ml, 60 ng/mL, 70 ng/mL, 80 ng/mL, 90 ng/ml, or 100 ng/mL dexamethasone). In some embodiments, the medium includes 60 ng/ml dexamethasone.

### Cell Populations

The methods described herein include agitating a plurality of cell populations to produce aggregates. Cell populations may be optimized to maintain the appropriate morphology, phenotype, and cellular function conducive to use in the methods of the disclosure. Cell populations of the methods described herein may include a population of mammalian (e.g., human) cells. The population may include primary cells, engineered cells, cell aggregates, induced pluripotent stem cell (iPSC)-derived cells, embryonic stem cell (ESC)-derived cells, transdifferentiated cells, or a combination thereof. The cells may be primary cells, e.g., primary cells that are expanded in vitro. In some embodiments, the population of cells includes endocrine, exocrine, paracrine, heterocrine, autocrine, or juxtacrine cells.

In some embodiments, the population of cells includes Leydig cells, adrenal cortical cells, pituitary cells, thyrocytes, granulosa cells, mammary gland epithelial cells, thymocytes, thymic epithelial cells, hypothalamus cells, skeletal muscle cells, smooth muscle cells, and/or neuronal cells.

In some embodiments, the pituitary cells comprise thyrotropic pituitary cells, lactotropic pituitary cells, corticotropic pituitary cells, somatotropic pituitary cells, and/or gonadotropic pituitary cells.

In some embodiments, the neuronal cells comprise dopaminergic cells.

In some embodiments, the population of cells includes parenchymal cells (e.g., hepatocytes, pancreatic exocrine cells, myocytes, pancreatic endocrine cells, neurons, enterocytes, adipocytes, splenic cells, kidney cells, biliary cells, Kupffer cells, stellate cells, cardiac muscle cells, alveolar cells, bronchiolar cells, club cells, urothelial cells, mucous cells, parietal cells, chief cells, G cells, goblet cells, enteroendocrine cells, Paneth cells, M cells, tuft cells, glial cells, gall bladder cells, keratinocytes, melanocytes, Merkel cells, Langerhans cells, osteocytes, osteoclasts, esophageal cells, photoreceptor cells, and corneal epithelial cells). In some embodiments, the parenchymal cells are pancreatic cells (e.g., alpha, beta, gamma, delta, epsilon cells, or any combination thereof). In some embodiments, the parenchymal cells include beta cells.

In some embodiments, the methods include aggregating two or more populations of cells (e.g., two, three, four, five, six, seven, eight, nine, ten, or more populations of cells).

In some embodiments, the population of cells includes a population of hepatocytes and a population of stromal cells.

In some embodiments, the stromal cells include fibroblasts. In some embodiments, the fibroblasts are human dermal fibroblasts (e.g., normal human dermal fibroblasts, neonatal foreskin fibroblasts, human lung fibroblasts, human ventricular cardiac fibroblasts, human atrial cardiac fibroblasts, human uterine fibroblasts, human bladder fibroblasts, human gingival fibroblasts, human pericardial fibroblasts, human gall bladder fibroblasts, human portal vein fibroblasts, human vas deferens fibroblasts). In some embodiments, the fibroblasts are human dermal fibroblasts. In some embodiments, the fibroblasts are normal human dermal fibroblasts. In some embodiments, the fibroblasts are neonatal foreskin fibroblasts. In some embodiments, the fibroblasts human lung fibroblasts. In some embodiments, the fibroblasts are human ventricular cardiac fibroblasts. In some embodiments, the fibroblasts are human atrial cardiac fibroblasts. In some embodiments, the fibroblasts are human uterine fibroblasts. In some embodiments, the fibroblasts are human bladder fibroblasts. In some embodiments, the fibroblasts are human gingival fibroblasts. In some embodiments, the fibroblasts are human pericardial fibroblasts. In some embodiments, the fibroblasts are human gall bladder fibroblasts. In some embodiments, the fibroblasts are human portal vein fibroblasts. In some embodiments, the fibroblasts are vas deferens fibroblasts.

In some embodiments, the cells are engineered cells, primary cells, or transdifferentiated cells.

In some embodiments, the engineered cells are engineered to express or secrete a protein, such as an antibody, a cytokine, an enzyme, a coagulation factor, or a hormone. The protein may be, for example, an endogenous human protein or an engineered protein.

In some embodiments, the first population of cells includes stromal cells (e.g., fibroblasts). The fibroblasts may be, e.g., primary fibroblasts, induced pluripotent stem cell (iPSC)-derived fibroblasts, or embryonic stem cell (ESC)-derived fibroblasts. In some embodiments, the fibroblasts are genetically engineered fibroblasts.

In some embodiments, the second population of cells includes parenchymal cells. The parenchymal cells may be, e.g., hepatocytes (e.g., human hepatocytes, e.g., primary human hepatocytes) or hepatocyte precursor cells. The hepatocytes may be, e.g., primary hepatocytes (e.g., primary human hepatocytes), iPSC-derived hepatocytes, or ESC-derived hepatocytes. The hepatocytes may be genetically engineered hepatocytes. The parenchymal cells may include pancreatic cells (e.g., alpha, beta, gamma, delta, or epsilon cells, or a combination thereof) or pancreatic precursor cells. The pancreatic cells may be, e.g., primary human pancreatic cells, iPSC-derived pancreatic cells, or ESC-derived pancreatic cells. In some embodiments, the pancreatic cells are genetically engineered pancreatic cells

In some embodiments, the first population of cells includes fibroblasts, and the second population of cells includes hepatocytes.

In some embodiments, the first population of cells includes fibroblasts, and the second population includes hepatocyte precursor cells.

In some embodiments, the first population of cells includes fibroblasts, and the second population includes pancreatic beta cells.

In some embodiments, a ratio of the first population of cells (e.g., stromal cells, e.g., fibroblasts, e.g., normal human dermal fibroblasts) to the second population of cells (e.g., parenchymal cells, e.g., hepatocytes, e.g., primary human hepatocytes) is from 10:1 to 1:10 (e.g., 10:1 to 1:1, 9:1 to 1:1, 8:1 to 1:1, 7:1 to 1:1, 6:1 to 1:1, 5:1 to 1:1, 4:1 to 1:1, 3:1 to 1:1, 2:1 to 1:1, 1:10 to 1:1 to 1:2, 1:1 to 1:3, 1:1 to 1:4, 1:1 to 1:5, 1:1 to 1:6, 1:1 to 1:7, 1:1 to 1:8, 1:1 to 1:9, or 1:1 to 1:10). For example, the ratio of the first population of cells to the second population of cells may be 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10. In some embodiments, the ratio is 2:1.

In some embodiments, the first population of cells (e.g., stromal cells, e.g., fibroblasts, e.g., normal human dermal fibroblasts) and the second population of cells (e.g., parenchymal cells, e.g., hepatocytes, e.g., primary human hepatocytes) do not expand (e.g., in cell number) by more than 30% during the agitating step. For example, in some embodiments, the first population of cells and the second population of cells do not expand by more than 30%, 25%, 20%, 15%, 10%, 9%, 8%, 7%, 6%, 5%, 4%, 3%, 2%, or 1%.

In some embodiments, the methods promote aggregation without causing a substantial increase in the number of cells. For example, in some embodiments, the ratio of the first population of cells to the second population of cells does not increase by more than 10-fold (e.g., does not increase by more than 9.5-fold, 9-fold, 8-fold, 7.5-fold, 7-fold, 6.5-fold, 6-fold, 5.5-fold, 5-fold, 4.5-fold, 4-fold, 3.5-fold, 3-fold, 2.5-fold, 2-fold, 1.5-fold, or 1-fold). For example, in some embodiments, the initial ratio of the first population of cells to the second population of cells (e.g., NHDF: PHH) is 2:1, and the ratio does not increase to more than 4:1 or 5:1 (i.e., an increase of no more than 2.5-fold).

In some embodiments, the ratio of the first population of cells to the second population of cells increases by 1-fold to 10-fold (e.g., 1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, or 10-fold, e.g., 1-fold to 3-fold, e.g., 1-fold to 2.5-fold, e.g., 1-fold, 1.5-fold, 2-fold, or 2.5-fold).

Fibroblasts may have a double time of 24 hours to 36 hours. Accordingly, in some embodiments, an aggregation time of 24 hours to 36 hours (e.g., 24 hours) is at or below the doubling time for fibroblasts, thus precluding any increase in cell number during aggregation. Similarly, PHH have a very large doubling time, e.g., approaching infinity, that would also preclude any increase in cell number of PHH during aggregation.

In some embodiments, a ratio of the first population of cells (e.g., stromal cells, e.g., fibroblasts, e.g., normal human dermal fibroblasts) to the second population of cells (e.g., parenchymal cells, e.g., hepatocytes, e.g., primary human hepatocytes) is from 0.1:1 to 5:1. In some embodiments, the ratio is 0.2:1 to 3.8:1. In some embodiments, the ratio is 3.8:1. In some embodiments, the ratio is 3:1. In some embodiments, the ratio is 2.5:1. In some embodiments, the ratio is 2:1. In some embodiments, the ratio is 1:1. In some embodiments, the ratio is 0.2:1. In some embodiments, the first population of cells are present at a density of 1×10⁴ cells/mL to 1×10⁸ cells/mL. For example, the first population of cells may be present at a density of 1×10⁴ cells/mL to 1×10⁵ cells/mL (e.g., 1×10⁴ cells/mL, 2×10⁴ cells/mL, 3×10⁴ cells/mL, 4×10⁴ cells/mL, 5×10⁴ cells/mL, 6×10⁴ cells/mL, 7×10⁴ cells/mL, 8×10⁴ cells/mL, 9×10⁴ cells/mL, or 1×10⁵ cells/mL), 1×10⁵ cells/mL to 1×10⁶ cells/mL (e.g., 1×10⁵ cells/mL, 2×10⁵ cells/mL, 3×10⁵ cells/mL, 4×10⁵ cells/mL, 5×10⁵ cells/mL, 6×10⁵ cells/mL, 7×10⁵ cells/mL, 8×10⁵ cells/mL, 9×10⁵ cells/mL, or 1×10⁶ cells/mL), 1×10⁶ cells/mL to 1×10⁷ cells/mL (e.g., 1×10⁶ cells/mL, 2×10⁶ cells/mL, 3×10⁶ cells/mL, 4×10⁶ cells/mL, 5×10⁶ cells/mL, 6×10⁶ cells/mL, 7×10⁶ cells/mL, 8×10⁶ cells/mL, 9×10⁶ cells/mL, or 1×10⁷ cells/mL), or 1×10⁷ cells/mL to 1×10⁸ cells/mL (e.g., 1×10⁷ cells/mL, 2×10⁷ cells/mL, 3×10⁷ cells/mL, 4×10⁷ cells/mL, 5×10⁷ cells/mL, 6×10⁷ cells/mL, 7×10⁷ cells/mL, 8×10⁷ cells/mL, 9×10⁷ cells/mL, or 1×10⁸ cells/mL).

In some embodiments, the first population of cells includes fibroblasts (e.g., primary fibroblasts, e.g., primary human fibroblasts) and the density of the fibroblasts is 1×10⁵ cells/mL to 1×10⁷ cells/mL (e.g., 1×10⁵ cells/mL, 2×10⁵ cells/mL, 3×10⁵ cells/mL, 4×10⁵ cells/mL, 5×10⁵ cells/mL, 6×10⁵ cells/mL, 7×10⁵ cells/mL, 8×10⁵ cells/mL, 9×10⁵ cells/mL, 1×10⁶ cells/mL, 2×10⁶ cells/mL, 3×10⁶ cells/mL, 4×10⁶ cells/mL, 5×10⁶ cells/mL, 6×10⁶ cells/mL, 7×10⁶ cells/mL, 8×10⁶ cells/mL, 9×10⁶ cells/mL, or 1×10⁷ cells/mL). For example, the density of the fibroblasts may be, e.g., 6×10⁵ cells/mL.

In some embodiments, the second population of cells are present at a density of 1×10⁴ cells/mL to 1×10⁸ cells/mL. For example, the second population of cells may be present at a density of 1×10⁴ cells/mL to 1×10⁵ cells/mL (e.g., 1×10⁴ cells/mL, 2×10⁴ cells/mL, 3×10⁴ cells/mL, 4×10⁴ cells/mL, 5×10⁴ cells/mL, 6×10⁴ cells/mL, 7×10⁴ cells/mL, 8×10⁴ cells/mL, 9×10⁴ cells/mL, or 1×10⁵ cells/mL), 1×10⁵ cells/mL to 1×10⁶ cells/mL (e.g., 1×10⁵ cells/mL, 2×10⁵ cells/mL, 3×10⁵ cells/mL, 4×10⁵ cells/mL, 5×10⁵ cells/mL, 6×10⁵ cells/mL, 7×10⁵ cells/mL, 8×10⁵ cells/mL, 9×10⁵ cells/mL, or 1×10⁶ cells/mL), 1×10⁶ cells/mL to 1×10⁷ cells/mL (e.g., 1×10⁶ cells/mL, 2×10⁶ cells/mL, 3×10⁶ cells/mL, 4×10⁶ cells/mL, 5×10⁶ cells/mL, 6×10⁶ cells/mL, 7×10⁶ cells/mL, 8×10⁶ cells/mL, 9×10⁶ cells/mL, or 1×10⁷ cells/mL), or 1×10⁷ cells/mL to 1×10⁸ cells/mL (e.g., 1×10⁷ cells/mL, 2×10⁷ cells/mL, 3×10⁷ cells/mL, 4×10⁷ cells/mL, 5×10⁷ cells/mL, 6×10⁷ cells/mL, 7×10⁷ cells/mL, 8×10⁷ cells/mL, 9×10⁷ cells/mL, or 1×10⁸ cells/mL).

In some embodiments, the second population of cells includes hepatocytes (e.g., primary hepatocytes, e.g., primary human hepatocytes) and the density of the hepatocytes is 1×10⁵ cells/mL to 1×10⁷ cells/mL (e.g., 1×10⁵ cells/mL, 2×10⁵ cells/mL, 3×10⁵ cells/mL, 4×10⁵ cells/mL, 5×10⁵ cells/mL, 6×10⁵ cells/mL, 7×10⁵ cells/mL, 8×10⁵ cells/mL, 9×10⁵ cells/mL, 1×10⁶ cells/mL, 2×10⁶ cells/mL, 3×10⁶ cells/mL, 4×10⁶ cells/mL, 5×10⁶ cells/mL, 6×10⁶ cells/mL, 7×10⁶ cells/mL, 8×10⁶ cells/mL, 9×10⁶ cells/mL, or 1×10⁷ cells/mL). For example, the density of the hepatocytes may be, e.g., 3×10⁵ cells/mL.

In some embodiments, the total number of cells (e.g., viable cells) are present at a density of 1×10⁴ cells/mL to 1×10⁸ cells/mL. For example, the second population of cells may be present at a density of 1×10⁴ cells/mL to 1×10⁵ cells/mL (e.g., 1×10⁴ cells/mL, 2×10⁴ cells/mL, 3×10⁴ cells/mL, 4×10⁴ cells/mL, 5×10⁴ cells/mL, 6×10⁴ cells/mL, 7×10⁴ cells/mL, 8×10⁴ cells/mL, 9×10⁴ cells/mL, or 1×10⁵ cells/mL), 1×10⁵ cells/mL to 1×10⁶ cells/mL (e.g., 1×10⁵ cells/mL, 2×10⁵ cells/mL, 3×10⁵ cells/mL, 4×10⁵ cells/mL, 5×10⁵ cells/mL, 6×10⁵ cells/mL, 7×10⁵ cells/mL, 8×10⁵ cells/mL, 9×10⁵ cells/mL, or 1×10⁶ cells/mL), 1×10⁶ cells/mL to 1×10⁷ cells/mL (e.g., 1×10⁶ cells/mL, 2×10⁶ cells/mL, 3×10⁶ cells/mL, 4×10⁶ cells/mL, 5×10⁶ cells/mL, 6×10⁶ cells/mL, 7×10⁶ cells/mL, 8×10⁶ cells/mL, 9×10⁶ cells/mL, or 1×10⁷ cells/mL), or 1×10⁷ cells/mL to 1×10⁸ cells/mL (e.g., 1×10⁷ cells/mL, 2×10⁷ cells/mL, 3×10⁷ cells/mL, 4×10⁷ cells/mL, 5×10⁷ cells/mL, 6×10⁷ cells/mL, 7×10⁷ cells/mL, 8×10⁷ cells/mL, 9×10⁷ cells/mL, or 1×10⁸ cells/mL). In some embodiments, the total number of cells (e.g., viable cells) are present at a density of 1×10⁵ cells/mL to 1×10⁷ cells/mL (e.g., 3×10⁵ cells/mL to 1.5×10⁶ cells/mL). In some embodiments, the total number of cells (e.g., viable cells) are present at a density of 1.5×10⁶ cells/mL.

### Methods of Encapsulation

The aggregates produced by a method described herein may further be encapsulated in a scaffold (e.g., a biocompatible scaffold). Such scaffolds of encapsulated cells may be used to form a graft (e.g., tissue graft) for various downstream uses, such as implantation into a subject (e.g., a human subject). The methods include providing population of aggregates of the plurality of cells populations (e.g., a first cell population and a second cell population) and a biocompatible scaffold. The method may further include introducing a polymerizing agent or cross-linking reagent to polymerize or cross-link the biocompatible scaffold, thereby encapsulating the population of aggregates, e.g., in the biocompatible scaffold. In other examples, the method may include incubating the aggregates for a time and under conditions sufficient to result in polymerization of the biocompatible scaffold without adding a polymerizing agent or cross-linking reagent. The aggregates may include, for example, two or more populations of cells (e.g., two, three, four, five, six, seven, eight, nine, ten, or more populations of cells). The aggregates may include, for example, a first population of cells and a second population of cells.

### Biocompatible Scaffolds

The aggregates described herein may be encapsulated in a biocompatible scaffold containing the cells, e.g., hepatocytes and stromal cells. In some embodiments, the population of cells (e.g., the population of hepatocytes and the population of stromal cells) are aggregated in spheroids. In some embodiments, the biocompatible scaffold has an x-axis, a y-axis, and a z-axis. For example, in some embodiments, the population of cells, e.g., hepatocytes and the optional population of stromal cells, are aggregated in spheroids and the spheroids are distributed non-homogenously, in a layer, along the z-axis of the biocompatible scaffold. In some embodiments, the spheroids are distributed homogenously along the x-axis of the biocompatible scaffold. In some embodiments, the spheroids are distributed homogenously along the y-axis of the biocompatible scaffold.

The biocompatible scaffold may be liquid, gel, semi-solid, or solid at room temperature (e.g., 25° C.). The biocompatible scaffold may be biodegradable or non-biodegradable. In some embodiments, the scaffold is bioresorbable or bioreplaceable. Exemplary biocompatible scaffolds include polymers and hydrogels include collagen, fibrinogen, fibrin, chitosan, MATRIGEL™, dextrans including chemically cross-linkable or photo-cross-linkable dextrans, processed tissue matrix such as submucosal tissue, PEG hydrogels (e.g., heparin-conjugated PEG hydrogels), poly (lactic-co-glycolic acid) (PLGA), hydroxyethyl methacrylate (HEMA), gelatin, alginate, agarose, polysaccharides, hyaluronic acid (HA), peptide-based self-assembling gels, thermo-responsive poly (NIPAAm). A number of biopolymers are known to those skilled in the art (Bryant and Anseth, J. Biomed. Mater. Res. (2002) 59 (1): 63-72; Mann et al., Biomaterials (2001) 22 (22): 3045-3051; Mann et al., Biomaterials (2001) 22 (5): 439-444, and Peppas et al., Eur. J. Pharm. Biopharm. (2000) 50 (1), 27-46; all incorporated by reference). In other embodiments, the biocompatible scaffold may contain a biopolymer having any of a number of growth factors, adhesion molecules, degradation sites or bioactive agents to enhance cell viability or for any of a number of other reasons. Such molecules are well known to those skilled in the art.

In some embodiments, the PEG hydrogel may be chemically cross-linkable and/or modified with bifunctional groups.

In certain embodiments, the biocompatible scaffold includes allogeneic components, autologous components, or both allogeneic components and autologous components. In certain embodiments, the biocompatible scaffold includes synthetic or semi-synthetic materials. In certain embodiments, the biocompatible scaffold includes a framework or support, such as a fibrin-derived scaffold.

In some embodiments, the biocompatible scaffold includes fibrin. In some embodiments, the polymerizing agent includes thrombin. Following introduction of a polymerizing agent (e.g., thrombin), the fibrinogen in the scaffold is polymerized into fibrin.

In some embodiments, the biocompatible scaffold further includes a reinforcing agent. For example, the reinforcing agent may include collagen, poly (ethylene glycol), polyvinylidene acetate (PVDA), polyvinylidene fluoride (PVDF), poly (lactic-co-glycolic) acid (PLGA), or poly (I-lactic acid) (PLLA).

Biocompatible hydrogel scaffolds suitable for use include any polymer that is gellable in situ, e.g., one that does not require chemicals or conditions (e.g., temperature or pH) that are not cytocompatible. This includes both stable and biodegradable biopolymers.

Polymers for use herein are preferably crosslinked, for example, ionically crosslinked. In certain embodiments, the methods and constructs described herein use polymers in which polymerization can be promoted photochemically (i.e., photo crosslinked), by exposure to an appropriate wavelength of light (i.e., photopolymerizable) or a polymer which is weakened or rendered soluble by light exposure or another stimulus. Although some of the polymers listed above are not inherently light sensitive (e.g., collagen, HA), they may be made light sensitive by the addition of acrylate or other photosensitive groups.

In certain embodiments, the method utilizes a photoinitiator. A photoinitiator is a molecule that is capable of promoting polymerization of hydrogels upon exposure to an appropriate wavelength of light as defined by the reactive groups on the molecule. In the context of the disclosure, photoinitiators are cytocompatible. A number of photoinitiators are known that can be used with different wavelengths of light. For example, 2,2-dimethoxy-2-phenyl-acetophenone, HPK 1-hydroxycyclohexyl-phenyl ketone and Irgacure 2959 (hydroxyl-1-[4-(hydroxyethoxy)phenyl]-2methyl-1 propanone) are all activated with UV light (365 nm). Other crosslinking agents activated by wavelengths of light that are cytocompatible (e.g., blue light) can also be used with the methods described herein.

In other embodiments, the method involves the use of polymers bearing non-photochemically polymerizable moieties. In certain embodiments, the non-photochemically polymerizable moieties are Michael acceptors. Non-limiting examples of such Michael acceptor moieties include α,β-unsaturated ketones, esters, amides, sulfones, sulfoxides, phosphonates. Additional non-limiting examples of Michael acceptors include quinines and vinyl pyridines. In some embodiments, the polymerization of Michael acceptors is promoted by a nucleophile. Suitable nucleophiles include, but are not limited to thiols, amines, alcohols, and molecules possessing thiol, amine, and alcohol moieties. In certain embodiments, the disclosure features use of thermally crosslinked polymers.

In some embodiments, the z-axis of the biocompatible scaffold is from 500 μm to 5 mm (e.g., 600 μm to 4 mm, 700 μm to 3 mm, 800 μm to 2 mm, or 900 μm to 1 mm). For example, in some embodiments, the z-axis of the biocompatible scaffold is from 600 μm to 4 mm. In some embodiments, the z-axis of the biocompatible scaffold is from 700 μm to 3 mm. In some embodiments, the z-axis of the biocompatible scaffold is from 800 μm to 2 mm. In some embodiments, the z-axis of the biocompatible scaffold is from 900 μm to 1 mm.

In some embodiments, the z-axis of the biocompatible scaffold is 500 μm. In some embodiments, the z-axis of the biocompatible scaffold is 600 μm. In some embodiments, the z-axis of the biocompatible scaffold is 700 μm. In some embodiments, the z-axis of the biocompatible scaffold is 800 μm. In some embodiments, the z-axis of the biocompatible scaffold is 900 μm. In some embodiments, the z-axis of the biocompatible scaffold is 1 mm. In some embodiments, the z-axis of the biocompatible scaffold is 2 mm. In some embodiments, the z-axis of the biocompatible scaffold is 3 mm. In some embodiments, the z-axis of the biocompatible scaffold is 4 mm. In some embodiments, the z-axis of the biocompatible scaffold is 5 mm.

In some embodiments, the ratio of height of the biocompatible scaffold to height of the layer of cell aggregates, e.g., hepatocyte and stromal cell aggregates, in the biocompatible scaffold is from 20:1 to 1:1 (e.g., 19:1 to 1:1, 18:1 to 1:1, 17:1 to 1:1, 16:1 to 1:1, 15:1 to 1:1, 14:1 to 1:1, 13:1 to 1:1, 12:1 to 1:1, 11:1 to 1:1, 10:1 to 1:1, 9:1 to 1:1, 8:1 to 1:1, 7:1 to 1:1, 6:1 to 1:1, 5:1 to 1:1, 4:1 to 1:1, 3:1 to 1:1, or 2:1 to 1:1). For example, in some embodiments the ratio of height of the biocompatible scaffold to height of the layer is from 19:1 to 1:1. In some embodiments, the ratio of height of the biocompatible scaffold to height of the layer is from 18:1 to 1:1. In some embodiments, the ratio of height of the biocompatible scaffold to height of the layer is from 17:1 to 1:1. In some embodiments, the ratio of height of the biocompatible scaffold to height of the layer is from 16:1 to 1:1. In some embodiments, the ratio of height of the biocompatible scaffold to height of the layer is from 15:1 to 1:1. In some embodiments, the ratio of height of the biocompatible scaffold to height of the layer is from 14:1 to 1:1. In some embodiments, the ratio of height of the biocompatible scaffold to height of the layer is from 13:1 to 1:1. In some embodiments, the ratio of height of the biocompatible scaffold to height of the layer is from 12:1 to 1:1. In some embodiments, the ratio of height of the biocompatible scaffold to height of the layer is from 11:1 to 1:1. In some embodiments, the ratio of height of the biocompatible scaffold to height of the layer is from 10:1 to 1:1. In some embodiments, the ratio of height of the biocompatible scaffold to height of the layer is from 9:1 to 1:1. In some embodiments, the ratio of height of the biocompatible scaffold to height of the layer is from 8:1 to 1:1. In some embodiments, the ratio of height of the biocompatible scaffold to height of the layer is from 7:1 to 1:1. In some embodiments, the ratio of height of the biocompatible scaffold to height of the layer is from 6:1 to 1:1. In some embodiments, the ratio of height of the biocompatible scaffold to height of the layer is from 5:1 to 1:1. In some embodiments, the ratio of height of the biocompatible scaffold to height of the layer is from 4:1 to 1:1. In some embodiments, the ratio of height of the biocompatible scaffold to height of the layer is from 3:1 to 1:1.

In some embodiments, the biocompatible scaffold includes a synthetic heparin mimetic. In particular, the synthetic polymer of the invention may include an amount of negative charge that, in some embodiments, is similar to the amount of negative charge present in heparin. Accordingly, the synthetic polymer of the disclosure can mimic the functional properties of heparin. For example, the synthetic polymer of the disclosure has the potential to bind various bioactive agents, e.g., growth factors, that naturally bind to heparin. Therefore, the synthetic polymer of the disclosure, as well as the hydrogel including the synthetic polymer described herein can bind various bioactive agents, e.g., growth factors, thereby preventing the bioactive agents from diffusing away and maintaining the bioactive agents at a high concentration locally, so that they can act on cells and promote various cell functions.

### EXAMPLES

### Example 1. Production of Aggregates in a Bioreactor

### VWB_005 Study

These experiments were performed to assess aggregation of normal human dermal fibroblast (NHDFs) and primary human hepatocytes (PHHs) in a vertical wheel bioreactor platform when seeded at same concentration as in a microwell platform that can be used to form aggregates. The impact of ROCK inhibitor, fibrinogen, and laminin were assessed on aggregation of NHDF and PHH in the VWB platform.

### Experimental Groups:

1. NHDF +PHH in ITS Only @ 36 RPM (75 mL volume)
2. NHDF +PHH in ITS+10 μM ROCK Inhibitor @ 36 RPM (75 mL volume)
3. NHDF +PHH in ITS+1 mg/mL FIBRYGAR @ 36 RPM (75 mL volume).
4. NHDF +PHH in ITS+5 μg/mL laminin @ 36 RPM (75 mL volume)
NHDF: seeded at 6e5 million cells/ml
PHH: seeded at 3.4e5 million cells/mL

A summary of the experimental conditions utilized is provided below.
1. Mixed NHDFs and PHHs together in a 500 ml conical by adding an equal volume of the PHH solution to the NHDF solution. e.g., if there were 100 ml of NHDFs, added 100 mL of PHH. This resulted in a 1.5× solution of NHDFs+PHHs. Target total combined volume: 200 mL minimum
2. Added 50 ml of the NHDF and PHH 1.5× mixture to each of the PBS-MINI® Chambers. Added ROCK Inhibitor, fibrinogen concentration (human), FIBRYGA®, or Laminin to corresponding chamber. Brought final volumes up to 75 mL with ITS Media.
3. Transferred the chambers to the bases set up in the incubators. Set each base to 36 RPM.

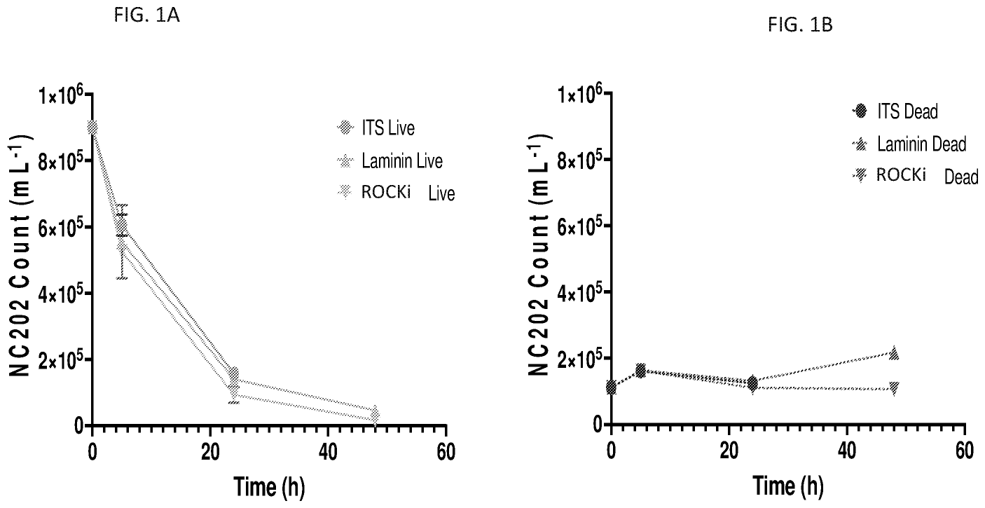

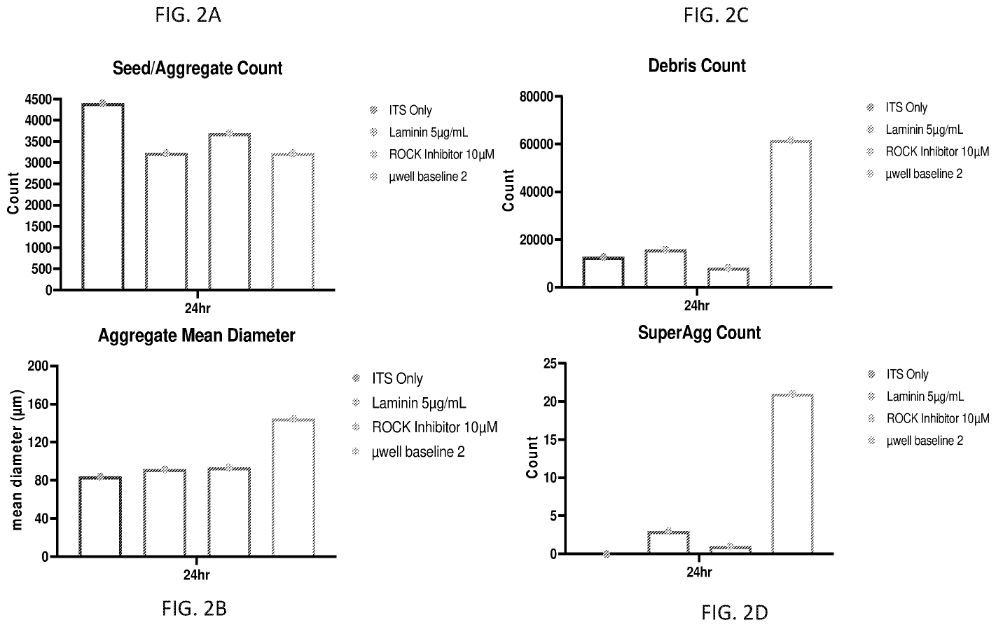

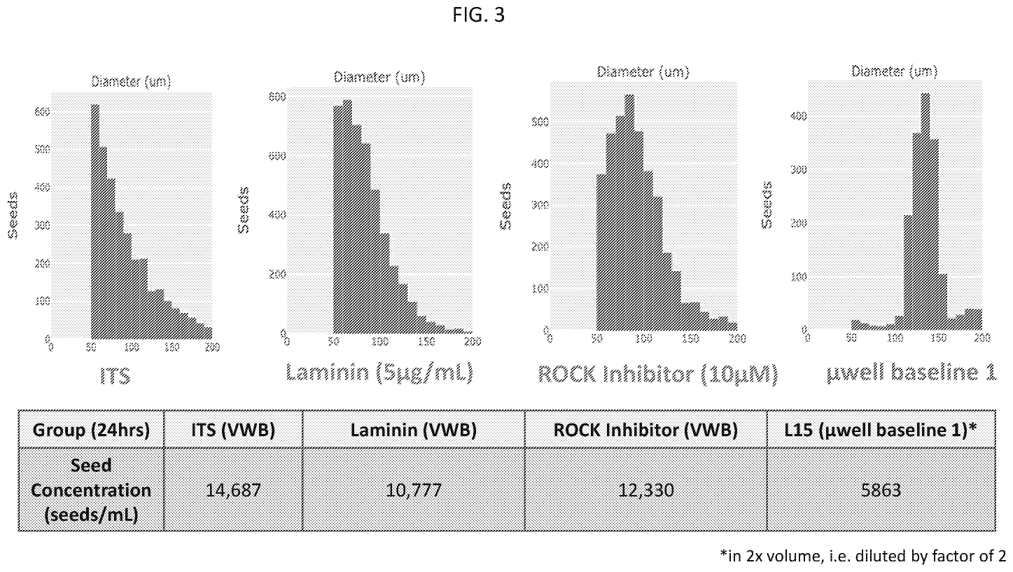

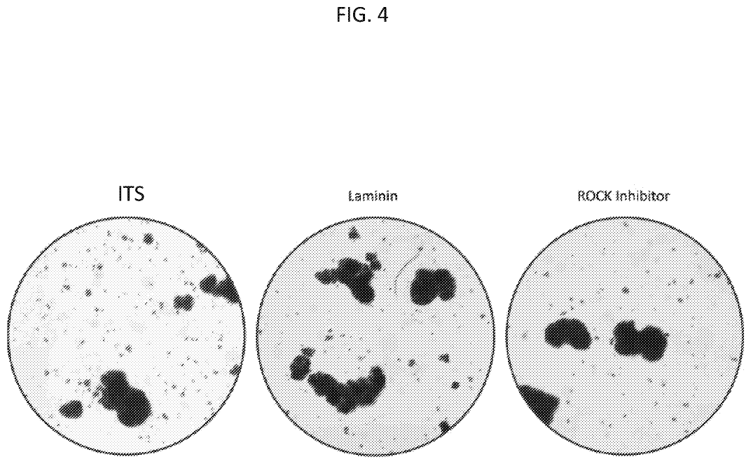

Across the ITS, Laminin, and ROCK Inhibitor conditions, the aggregate mean diameter and aggregate count were very similar; the differences appeared in debris and superaggegate count (FIGS. 1A, 1B, 2A-2D, 3, and 4). The ROCK inhibitor had less debris compared to the ITS and Laminin conditions. In terms of superaggregates count, all three conditions had low count (≤3) but the Laminin condition had the most superaggregates.

Superaggregates were identified as an optically opaque structure having a size of >200 μm and with an area/perimeter ratio of >45.

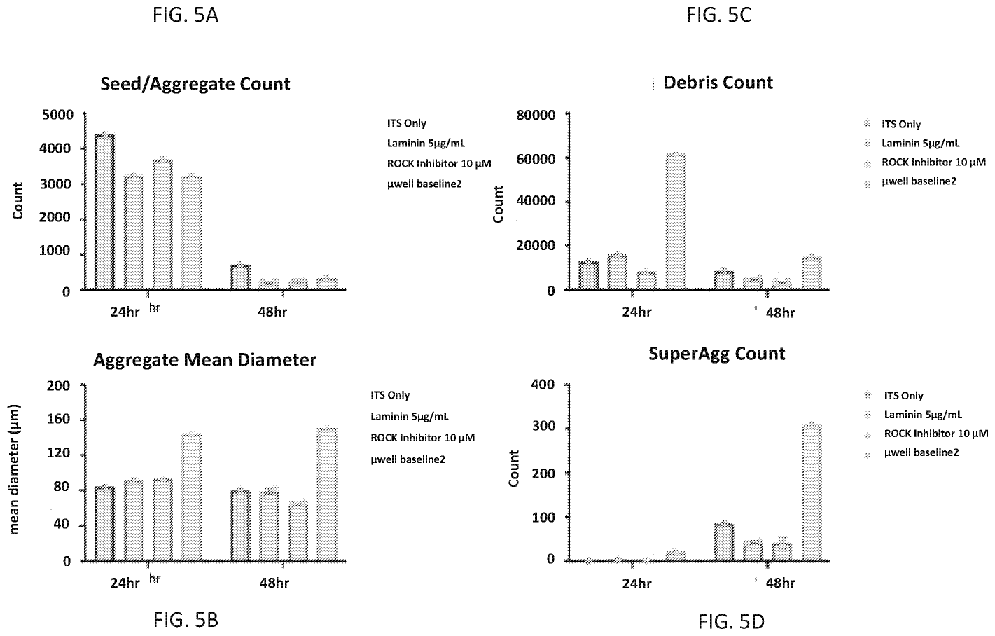

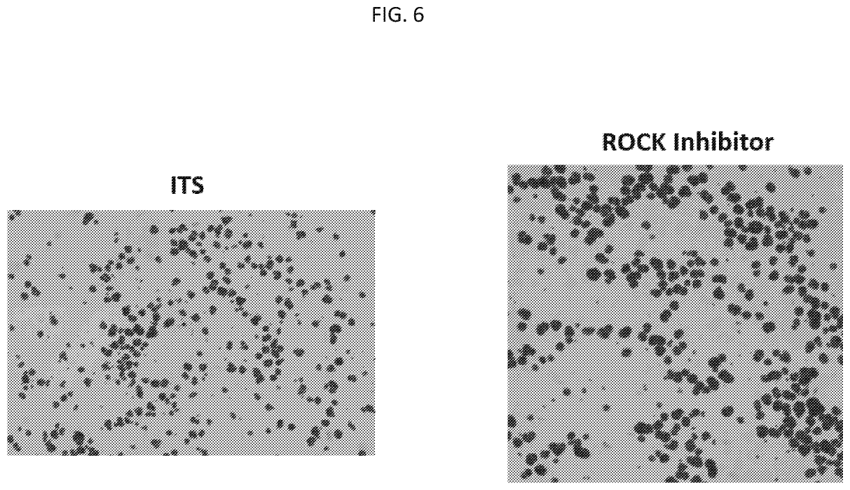

As shown in FIGS. 5A-5D and 6, aggregates formed (FIG. 6) at 24 hours and 48 hours. The number of aggregates decreased at 48 hours. The microwell baseline condition is a comparison condition in which cells were aggregated in microwells. The microwell condition produced more debris and superaggregates (FIGS. 5A-5D).

The VWB_005 study shows that NHDFs and PHHs, when seeded into the VWB platform, form aggregates, (also referred to as seeds) in the ITS, laminin (5 μg/mL) and ROCK inhibitor (10 μM) conditions.

### VWB_006 Study

Next, the potency of the seeds was assessed of via an ammonia clearance assay (IDEXX) and albumin secretion assay.

TABLE 1
Experimental Groups:

| | NHDF | PHH | | | |
|---|---|---|---|---|---|
| Group | (cells/mL) | (cells/mL) | Total Vol. | Passivation | ROCKi |
| A | 6e+5 | 3e+5 | 75 | Y | 0 μM |
| B | 6e+5 | 3e+5 | 75 | Y | 10 μM |

| | | | |
|---|---|---|---|
| C | 6e+5 | 3e+5 | Microwell study |

### Seed NHDFs and PHHs in Bioreactors

1. Mixed NHDFs and PHHs together in a 500 ml conical by adding an equal volume of the PHH solution to the NHDF solution. Target total combined volume: 100 mL minimum. Combined 67.4 mL of each cell mixture
2. Added 50 mL of the NHDF and PHH 1.5× mixture to both of the PBS-MINI® Chambers. Added ROCK Inhibitor to designated chamber. Brought final volumes up to 75 mL with ITS Media.
3. Transferred the chambers to the bases set up in the incubators. Set each base to 36 RPM.

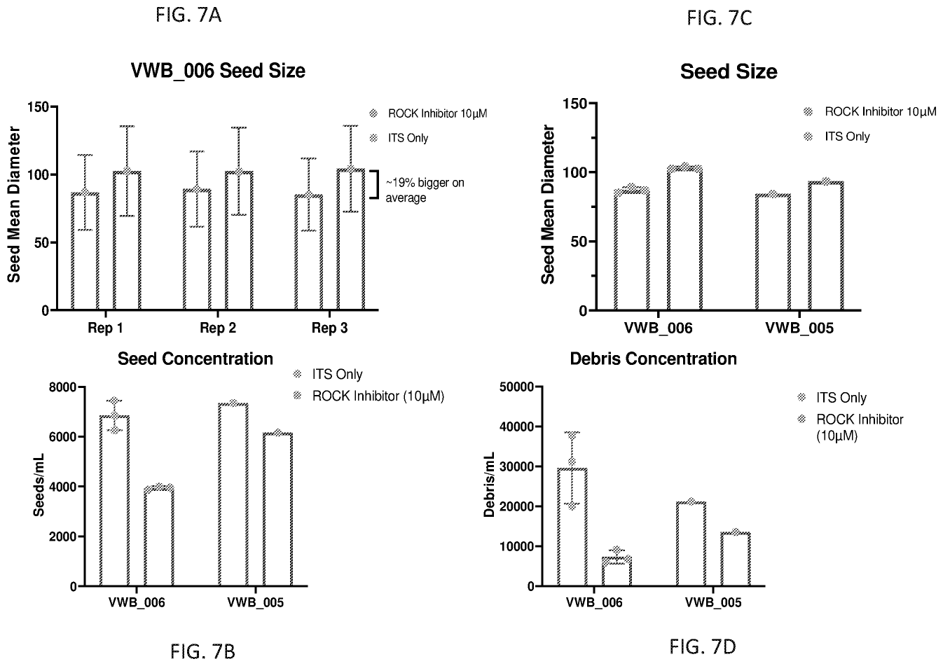

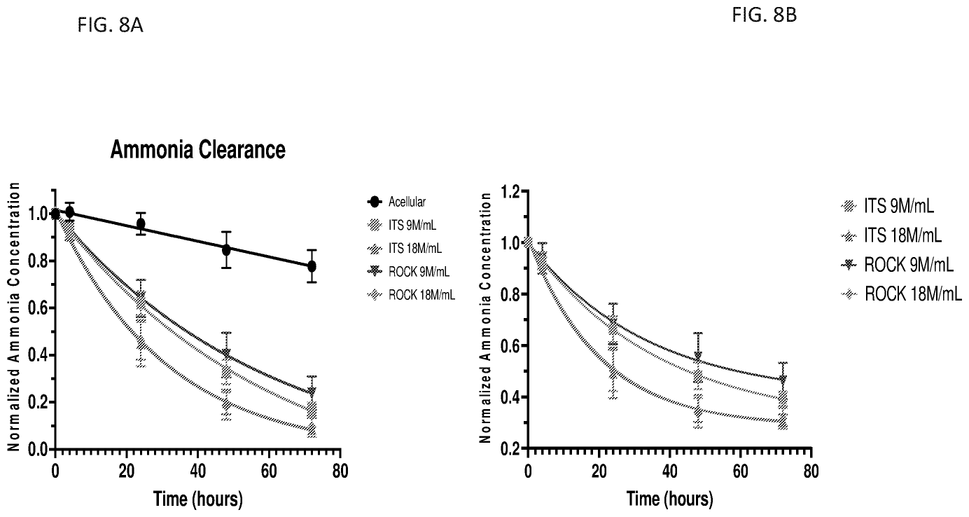

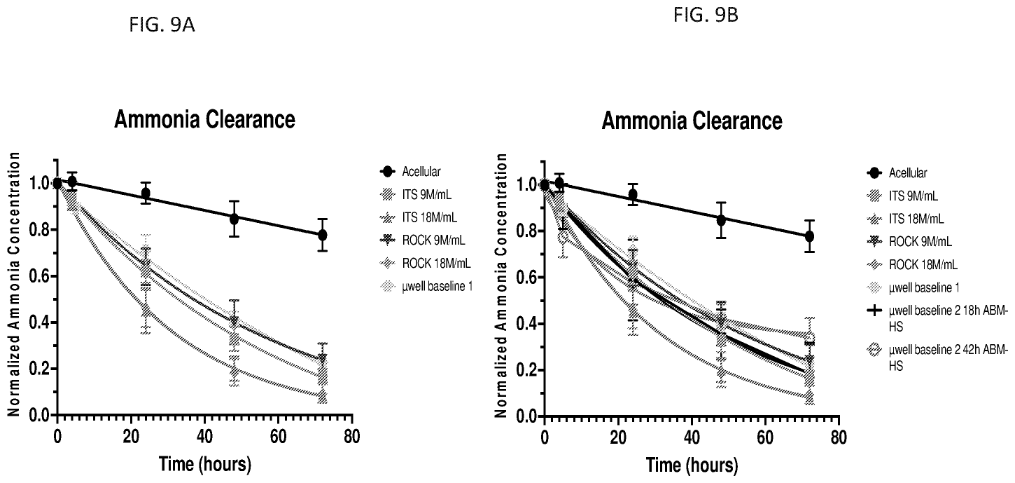

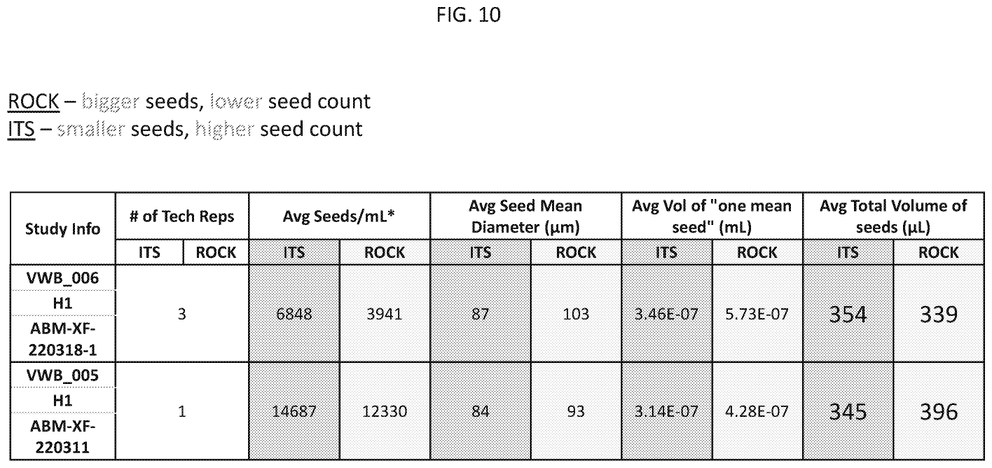

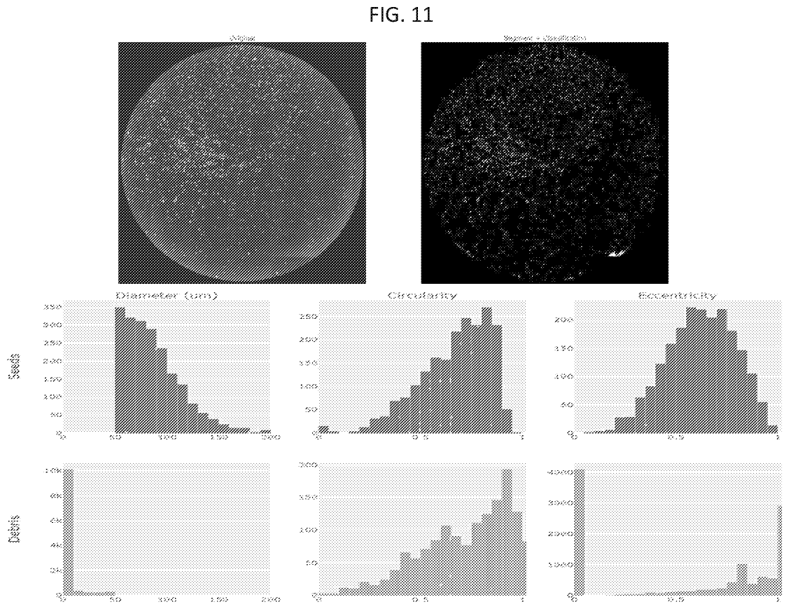

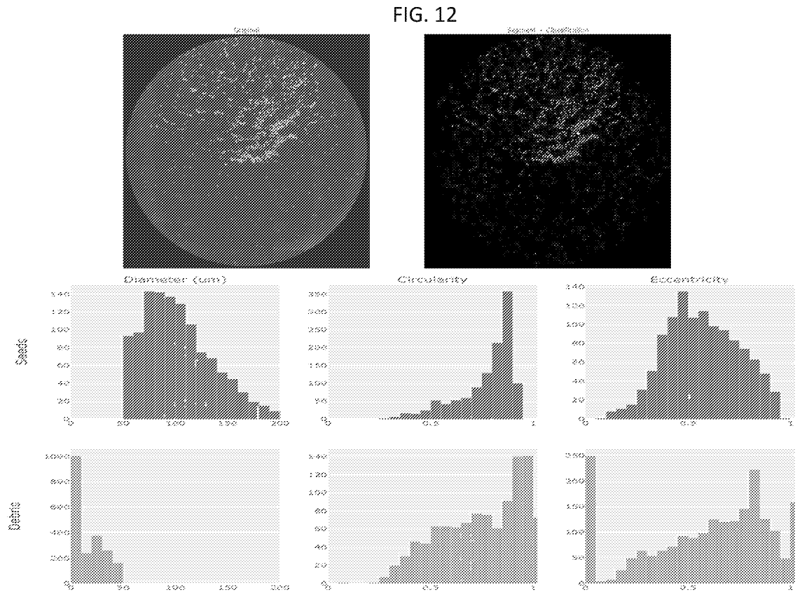

As shown in FIGS. 7A-7D, 10, 11, and 12, seeds were formed with ITS only or in the presence of ROCK inhibitor. In an ammonia clearance assay, these aggregates were able to reduce ammonia (FIGS. 8A and 8B) and showed comparable results to microwell based aggregates (FIGS. 9A and 9B)

In conclusion, aggregates or seeds were generated in the VWB platform, demonstrating proof-of-concept for suspension culture for aggregation. The VWB seeds were smaller than the ones generated in the microwell platform, but there was also significantly less debris and superaggregates compared to the microwell platform.

The 24-hour aggregation time was more successful than the 48 hour aggregation; in this experiment, 48 hours appeared to be too long and resulted in significant superaggregation formation. Out of the conditions tested, the ITS, Laminin and ROCK Inhibitor conditions produced positive and similar results between the three, with the ROCK inhibitor condition producing the lowest debris count at 24 hours.

### VWB_007 Study

In VWB_007, it was assessed whether aggregation can occur as in VWB_005 and VWB_006 with alternate media formulations suitable for good manufacturing process (GMP) grade. The first aim was to remove the animal derived component Fetal Bovine Serum (FBS) currently included in the aggregation media. This study tested three concentrations of human serum, and two concentrations of human platelet lysate to compare to base media condition. All media contained high levels of glucose. As there is potential that the cells will become more stressed in the lower serum conditions, ROCK inhibitor (ROCKi) was used in all conditions in this study.

TABLE 2
Experimental Groups:

| | NHDF | PHH | | | Dexameth- | |
|---|---|---|---|---|---|---|
| | (cells/ | (cells/ | Total | | asone | |
| Group | mL) | mL) | Vol. | Media | And Glucose | ROCKi |
| A | 6e+5 | 3e+5 | 75 | ITS (10% | Y | Y |
| | | | | FBS) | | |
| B | 6e+5 | 3e+5 | 75 | hgDMEM + | Y | Y |
| | | | | 1% HS | | |
| C | 6e+5 | 3e+5 | 75 | hgDMEM + | Y | Y |
| | | | | 5% HS | | |
| D | 6e+5 | 3e+5 | 75 | hgDMEM + | Y | Y |
| | | | | 10% HS | | |
| E | 6e+5 | 3e+5 | 75 | hgDMEM + | Y | Y |
| | | | | 1% PG | | |
| F | 6e+5 | 3e+5 | 75 | hgDMEM + | Y | Y |
| | | | | 5% PG | | |

### Seed NHDFs and PHHs in Bioreactors

For each condition:
A. Mixed NHDFs and PHHs together in the PHH 50 mL conical by adding an equal volume (20 mL) of the NHDF solution to the PHH solution (20 mL). Pipetted up and down thoroughly to mix the cells.
B. Transferred the NHDF and PHH cell mixture to the matching PBS-MINI® 0.1 L consumable (40 mL total).
C. Brought final volume up to 70 mL
   Transferred the chambers to the bases set up in the incubators. Set each base to 36 RPM.

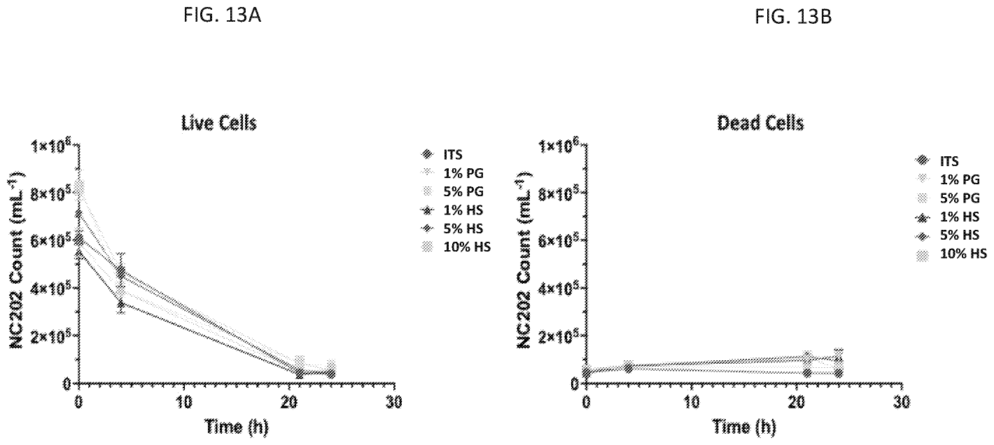

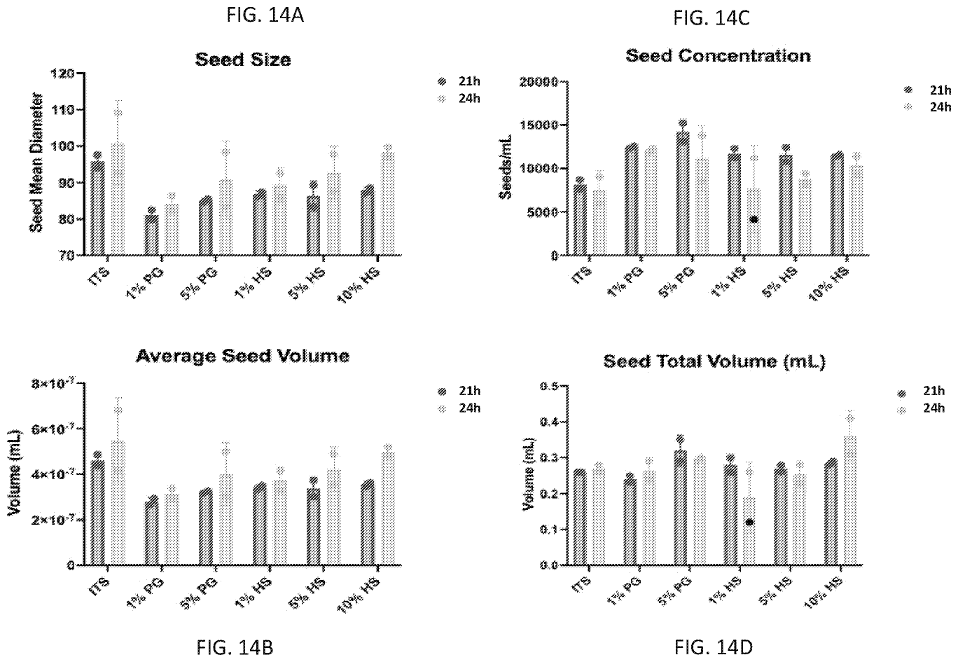

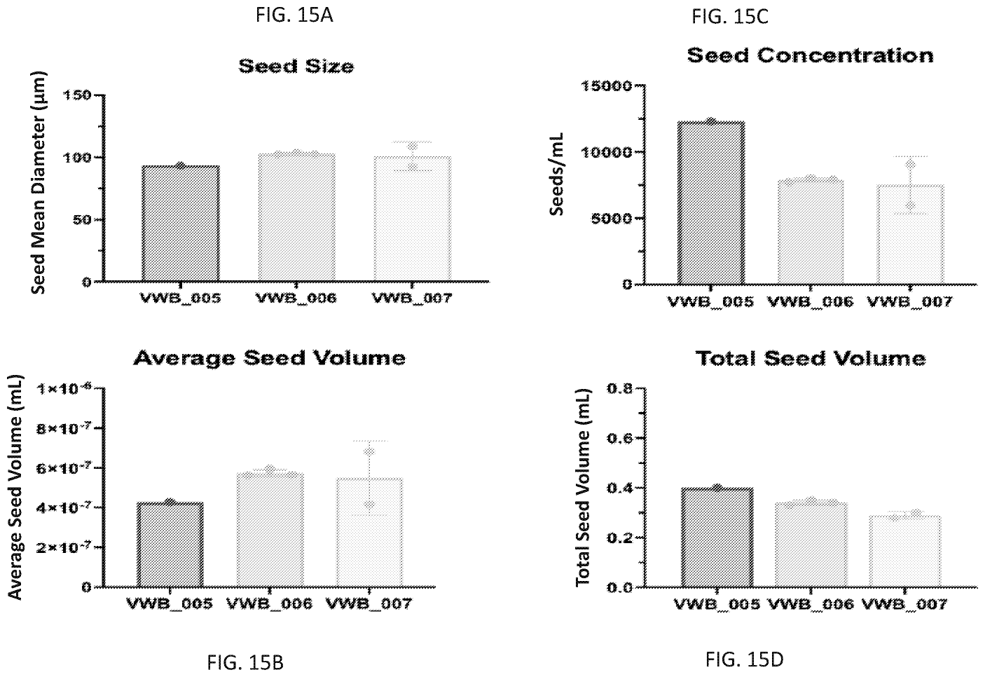

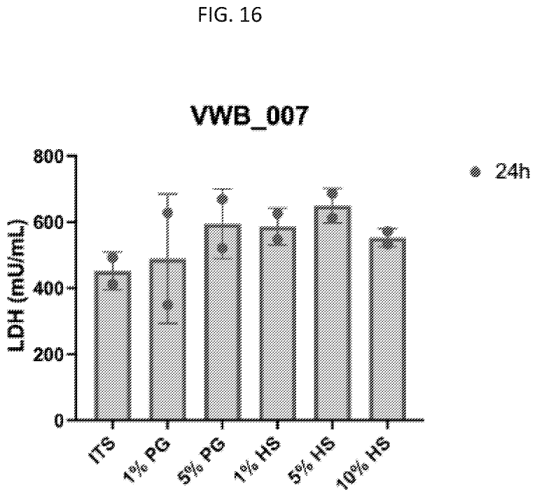

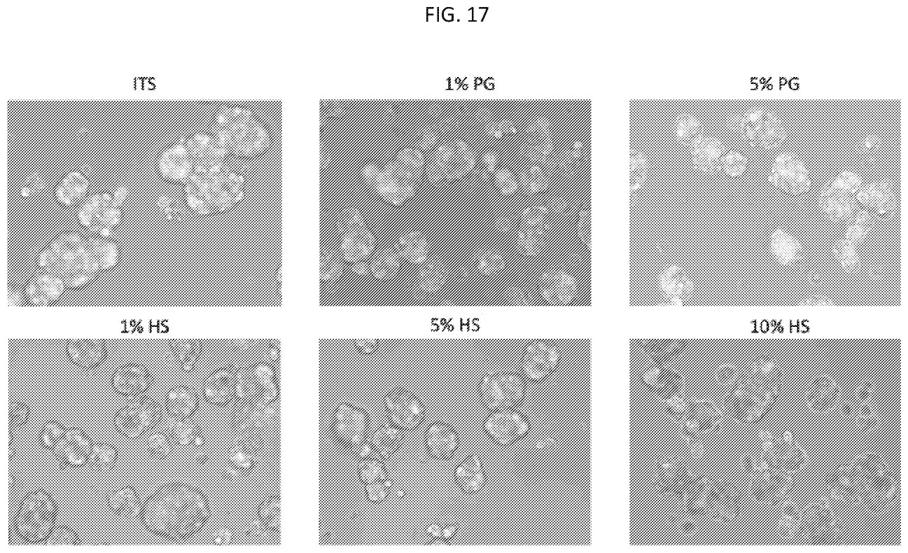

Similar to results seen in previous studies, the live cell concentration decreased over time and the dead cell concentration stayed relatively constant. This indicates that the single cells are being incorporated into aggregates over time, and not dying off (FIGS. 13A and 13B). FIG. 13A shows live cells, and FIG. 13B shows dead cells. As shown in FIGS. 14A-14D, 15A-15D, 16, and 17, aggregates were formed under various conditions with human serum (HS) and PLATELET GOLD™ (PG; human platelet lysate) with data comparable to aggregate size, concentration, average volume, and total volume as conditions without human serum or platelet lysate (FIGS. 14A-14D, 15A-15D, 16, and 17).

### Aggregate Washing

Individual vials of fibroblasts and hepatocytes were thawed and resuspended in cell culture media separately before a centrifugation-based wash step. After aggregation, the aggregates were concentrated using a centrifuge and formulated in a fibrinogen solution for encapsulation. This experiment evaluated if the CTS ROTEA™ Counterflow Centrifugation System was a viable option for the single cell washing after thaw, and the seed washing and formulation after aggregation.

### NHDF Thaw and Wash

NHDF cells were tested for thawing and washing. The roughly 40M viable cells used corresponded to the smallest practical wash run in our aggregation framework, a single PBS-MINI® 0.1 single-use vertical wheel bioreactor loaded at 6e5/mL NHDF cells. The 35 mL of cells in NHDF media were loaded into a bag and the bag was welded onto the ROTEA™ port, and 47 mL was harvested in a different bag. The harvest setpoint was 50 mL, and the missing volume can be attributed to the tubing dead volume that was welded off but not collected (45 cm of 3 mm ID tubing accounts for 3.2 mL). Viability was 96% after recovery, and 87% of the viable cells were recovered after the wash. These results indicate that counter flow centrifugation systems such as ROTEA™ can be used to wash NHDF with minimal loss of viability.

The second part of the ROTEA™ experiment was the washing of PHH. Viability of the PHH suspension was measured prior to the bag loading, at the harvest bag point, and in the waste bag. We ran three trials using the same flow rate to centrifugation speed ratio but varying the flow rate to vary the shear stress exerted on the PHH.

The flow rate is a factor that may be varied to improve the overall viability of the harvest.

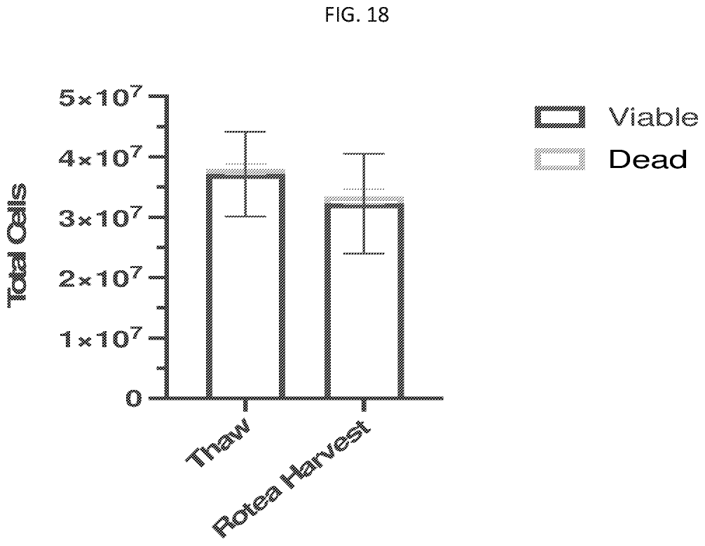

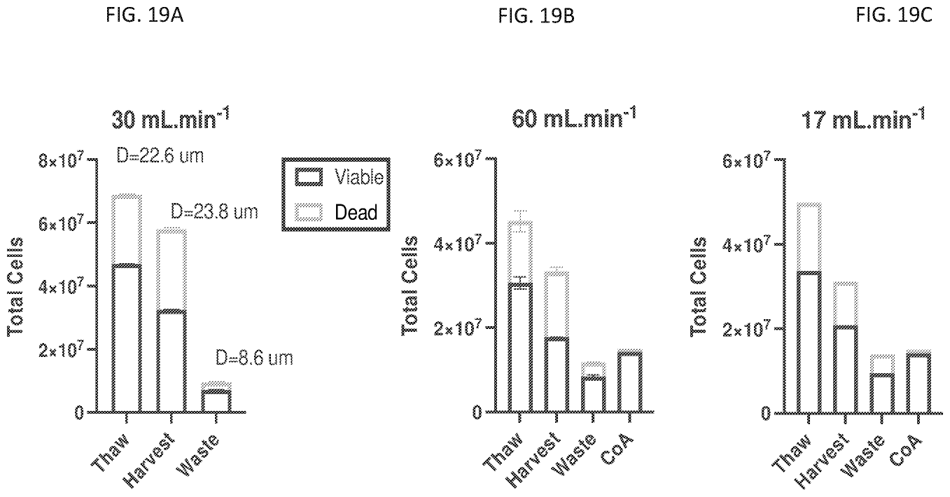

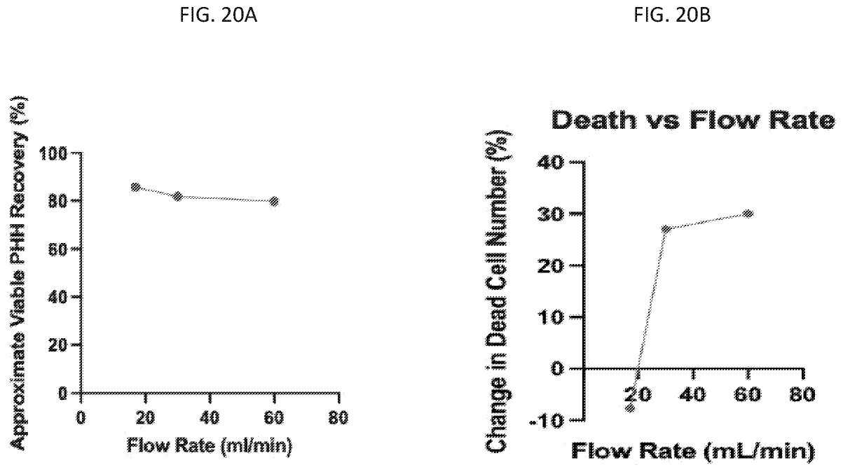

FIG. 18 shows the number of viable and dead cells (normal human dermal fibroblasts) after washing when thawed or washed with a CTS™ ROTEA™ harvest, which uses a counter flow centrifugation system. As shown in FIGS. 19A-19C, 20A, and 20B, show that the number of viable and dead PHH depended on different flow rates wash media.

### Seed Wash and Formulation

The aggregate washing and formulation pilot consisted of experiments on two separate days. On the first day, aggregates were prepared using a microwell approach and pooled for processing. The processing was split into two steps. First, the seeds were loaded into the fluid bed and returned to the initial bag for sampling. This approximates a simple wash recovery. Second, we aimed to exchange the buffer with 14 mg/ml of fibrinogen concentration (human) (FIBRYGA®), and harvest the concentrate. We notice two distinct populations of seeds and a debris count over 200,000.

The second day of seed washing used aggregates from VWB_007. The aggregates were pooled into two batches. Since the seeds generated in the suspension format are smaller than from the microwell format, the CTS™ ROTEA™ counter flow centrifugation system instrument settings were adjusted accordingly.

### Batch 1: ITS, 1% Human Serum, 5% Platelet Gold

The pool of the ITS, 1% Human Serum and 5% Platelet Gold seeds was measured using a customized application. For this trial the system output 7 mL of liquid (target 10 mL, 3 mL of air) into a syringe welded onto the harvest line. From a starting reactor harvest volume of 250 mL, this resulted in a 35× concentration.

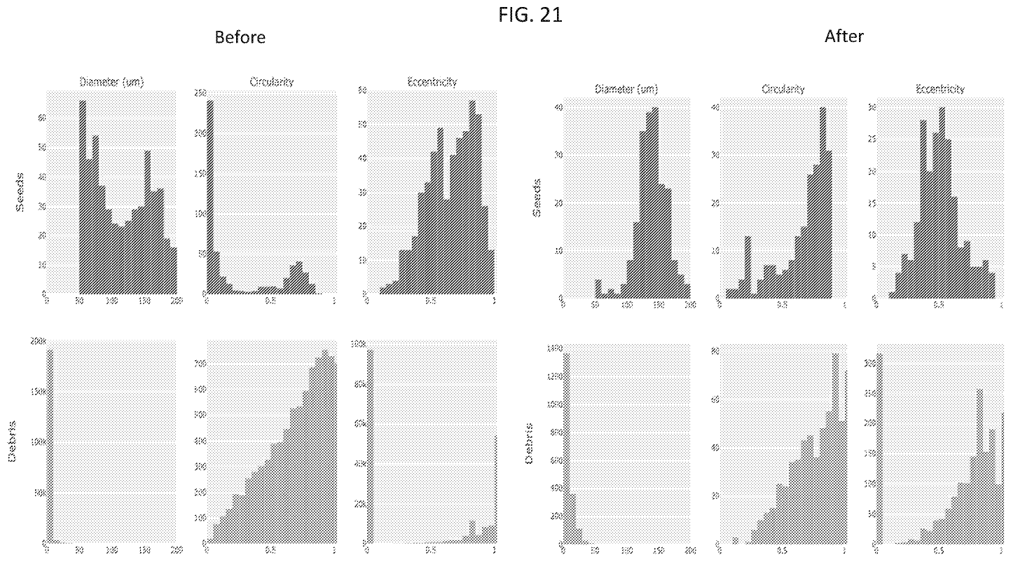

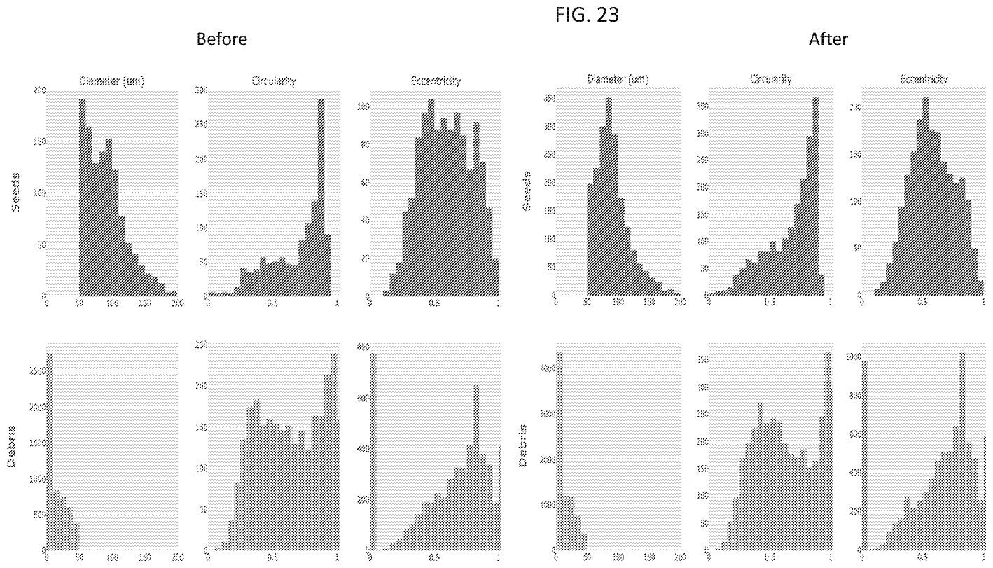

After the concentration and harvest the seeds diluted 40× and remeasured. Compared to the initial conditions, the seeds have a narrower size range and surprisingly there was a loss of the larger aggregates. The ROTEA™ can remove debris and result in more monodisperse aggregate populations (FIGS. 21 and 23).

### Batch 2: 1% Platelet Gold, 5% Human Serum, 10% Human Serum.

For the second batch, the flow rate was increased to attempt to achieve a higher purification and a more uniform fluid bed. The harvest volume setpoint was reduced to 5 mL. The pool of the 1% Platelet Gold, 5% Human Serum, 10% Human Serum reactors from VWB007 was measured.

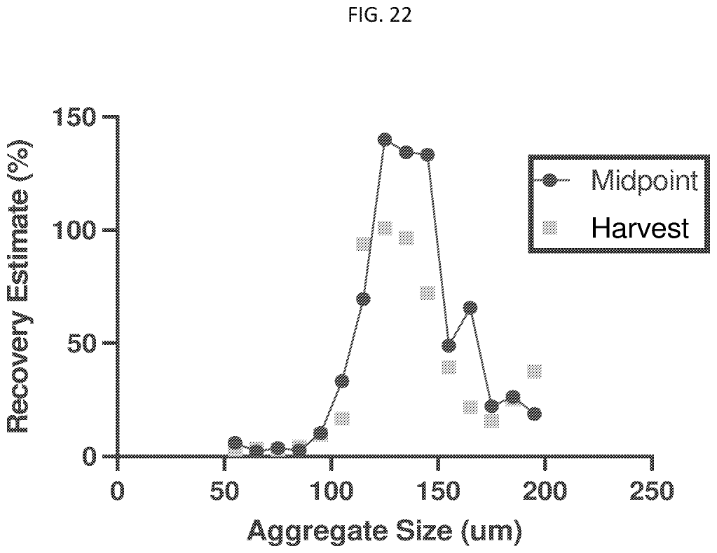

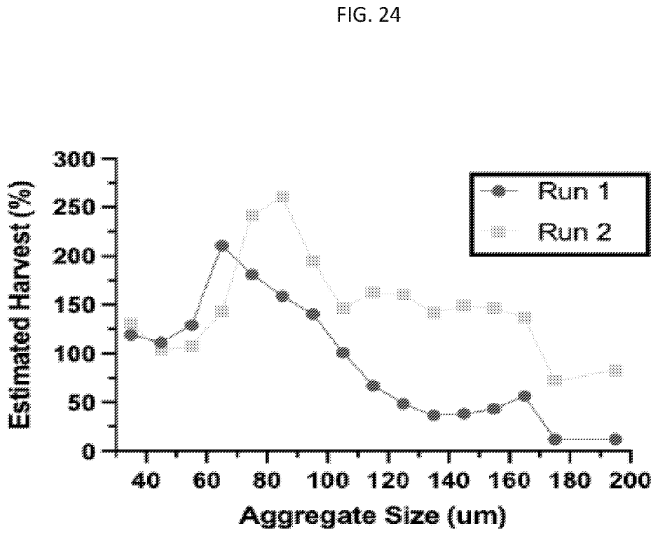

These results indicate that the ROTEA™ can be used to wash aggregates (seeds) as shown in FIGS. 22 and 24.

### VWB_010 Study

The VWB_005 Study described above showed that the VWB system can produce seeds. The VWB_006 Study described above, the seeds were encapsulated, their function was demonstrated via the ammonia clearance assay. Following the demonstration that the VWB system can produce functional seeds, this study evaluated whether the process could be translated to Good Manufacturing Practice (GMP) standards; in particular, using a GMP media formulation. In previous studies the aggregation media was ITS, which contains (nonhuman) animal derived supplements including Fetal Bovine Serum (FBS) and various other components from the ITS premix. In this study, a GMP-friendly media was formulated and generated that is devoid of all the GMP-noncompliant components. By substituting the ITS and FBS components with xenogeneic-free reagents, any potential risks of carrying animal-derived components are eliminated while ensuring the good health of the cells involved in the exemplary process described herein. Thus, Human Serum (HS) and Platelet Lysate (e.g., Platelet Gold (PG)) were investigated, since they are produced and packaged to GMP standards.

The VWB_007 Study described above tested aggregating with media made with varying percentages of Human Serum (HS) and Platelet Gold (PG) in comparison to media formulation containing 10% fetal bovine serum. Seeds formed in all conditions tested, supporting a transition over to a non-bovine source of serum in the aggregation media. In VWB_010 a subset of the media formulations queried in VWB_007 were tested, with the specific aim to understand the in vitro function of the seeds to clear ammonia.

NHDFs and PHHs were thawed from cryogenic storage, counted, and seeded at designated concentrations in a 2 NHDF: 1 PHH ratio in PBS MINI 0.1 L bioreactors. Four media conditions were tested: ITS (10% FBS), 10% Human Serum (HS), 5% HS, and 5% Platelet Gold (PG). The suspension cultures were allowed to run for 24 hours before harvest.

The readouts included microscopy, seed count analysis, lactate dehydrogenase (LDH), alanine transaminase (ALT)/aspartate transaminase (AST), and in vitro graft potency (ammonia clearance).

The major goals of this experiment were to assess aggregation of NHDFs and PHHs in the VWB platform when seeded in different media formulations. assess impact of changing serum type to xenogeneic-free (xeno-free) reagents on the VWB aggregation process and assess the potency and function of seeds aggregated in the VWB platform using xenogeneic-free aggregation media.

### Microscopy and Seed Count

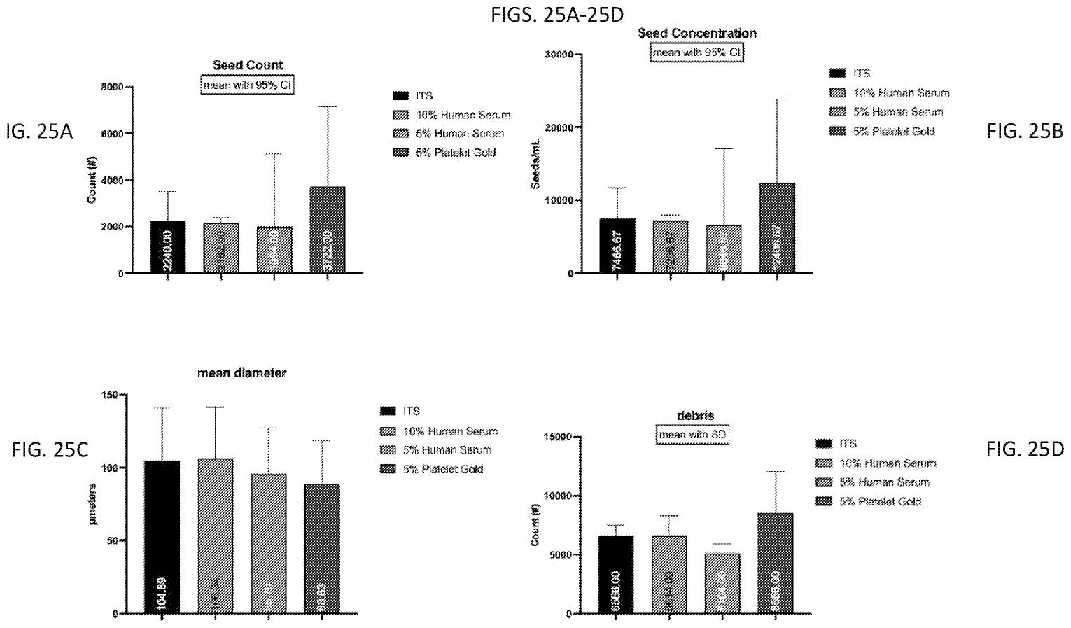

Images were captured after 24 hours of suspension driven aggregation. The samples were diluted 4× of their initial volume to analyze via computational analysis. FIGS. 25A-25D illustrate the results post computational analysis and include seed count, size, concentration, average seed volume (single seed), and total seed volume (volume fraction of seeds based on theoretical size).

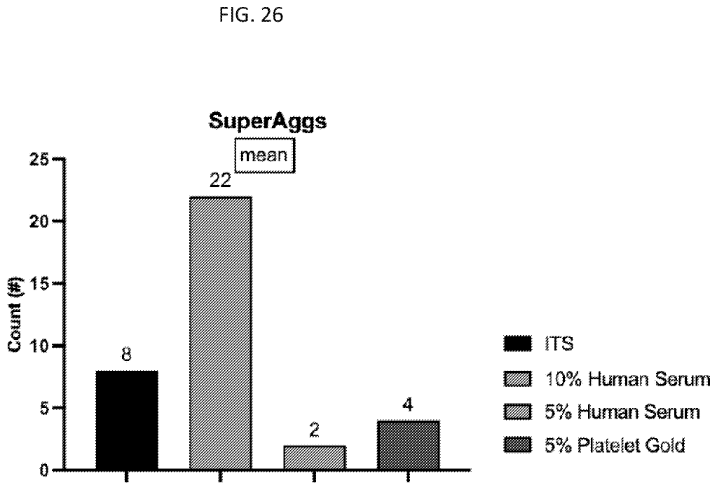

Across the four media conditions, the seed count was relatively similar around 2000-2200 seeds, but the 5% PG seeds showed higher counts (FIG. 25A). Translating the seed count to seed concentration, we counted the number of seeds per mL of reactor suspension. Again, the 5% PG condition was higher, meaning there are more seeds per mL compared to the other three conditions (FIG. 25B). The ITS and human serum conditions generated similar sized seeds, where the 5% PG condition generated smaller seeds on average out of all the tested groups (FIG. 25C). These data complement the earlier findings in FIG. 25B. There are more seeds per mL of suspension for the 5% PG condition, because the seeds are smaller. Seed size and debris are related. Larger seed conditions typically have less debris. This was seen in the ITS and 10% HS conditions (FIG. 25D). Surprisingly, the seeds formed using 5% HS were slightly smaller, but also resulted in less debris. 5% PG resulted in the most debris in the tested conditions. As shown in FIG. 26, super aggregates were highest in the 10% human serum condition.

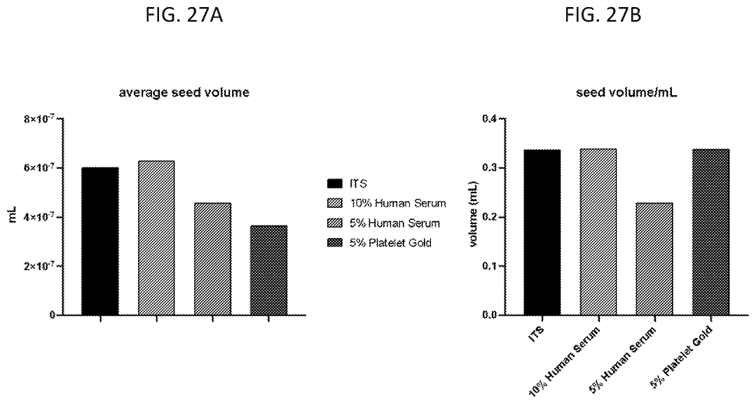

The seed mean diameter was very similar between Groups ITS and 10% HS, with 10% HS generating, on average, larger seeds. Groups 5% HS and 5% PG generated smaller seeds, with the 5% PG resulting in the smallest seeds out of all the Groups. As expected, the average seed volume followed a similar pattern and order. The total seed yield (seed volume/suspension volume) was approximately the same for Groups ITS, 10% HS and 5% PG (FIGS. 27A and 27B).

These data suggest that higher concentration of Human Serum generates similar seeds in terms of count, size, debris, and volume as the control condition which is the ITS Group, where the lower concentrated HS condition resulted in smaller seeds.

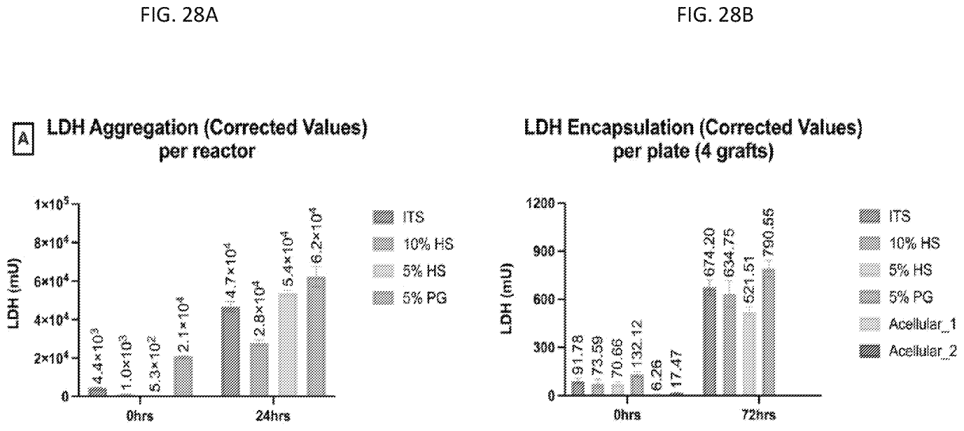

In FIGS. 28A and 28B, the cumulative amount of LDH computed for each vertical wheel reactor during aggregation (FIG. 28A) (1× Vertical wheel reactor) and the encapsulated seeds (n=4 grafts per media condition) is shown.

After 24 hours of aggregation, it appears that the 10% HS Group generated the least amount of LDH, followed by ITS and 5% PG, while the 5% HS Group generated the highest amount of LDH at time points of zero and 24 hours.

During encapsulation, it appears that the highest amount of LDH was generated by the 5% PG Group, the lowest value was for the 5% HS Group and the ITS & 10% HS Groups generated similar results.

The correction of these values was achieved by subtracting the values from the acellular samples.

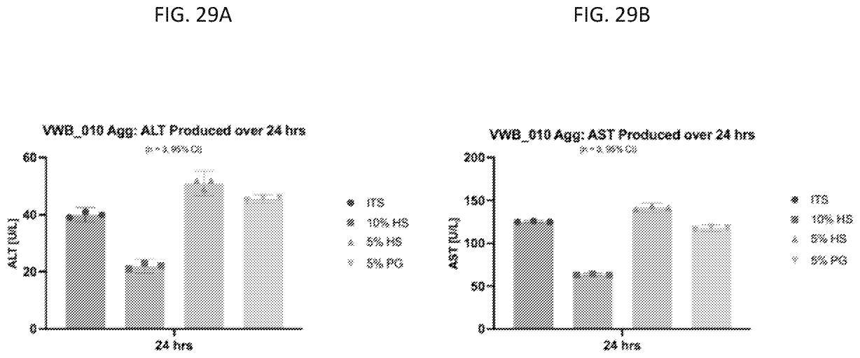

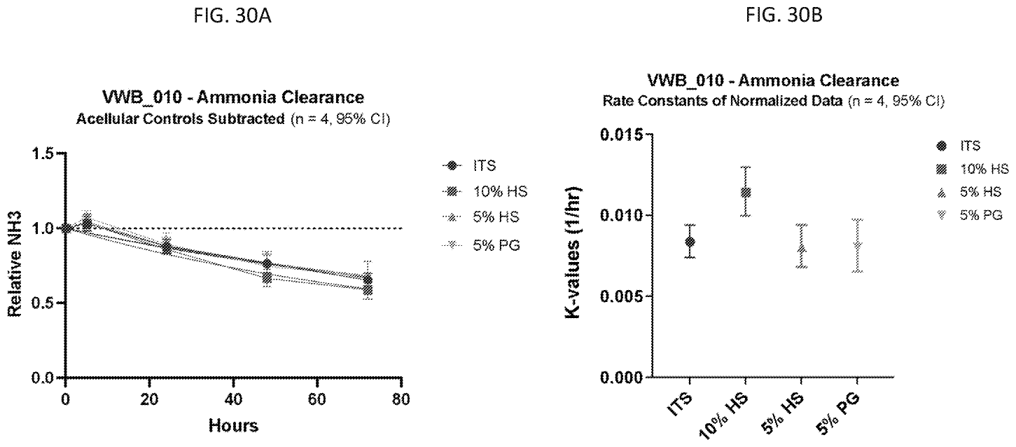

FIGS. 30A and 30B illustrate the ALT (FIG. 29A) and AST (FIG. 29B) values produced during the 24-hour aggregation period for the four different media groups. Out of the four media groups tested, ALT and AST levels during the 24-hour aggregation were lowest in the 10% HS group. ALT/AST levels in the 10% HS groups were approximately half of the levels seen in the ITS, 5% HS and 5% PG conditions.

This suggests that the primary human hepatocytes within the vertical wheel bioreactor are less stressed in the 10% HS formulation during the aggregation process compared to the control (ITS) and other test groups.

All tested media formulations produced seeds that were able to clear ammonia in the in vitro ammonia clearance assay. The groups performed very similarly with the 10% Human Serum group achieving the highest ammonia clearance out of all the tested Groups. These data indicate that switching to a xenogeneic-free source of serum in the aggregation media does not reduce ammonia clearance capabilities of the seeds (FIGS. 30A and 30B).

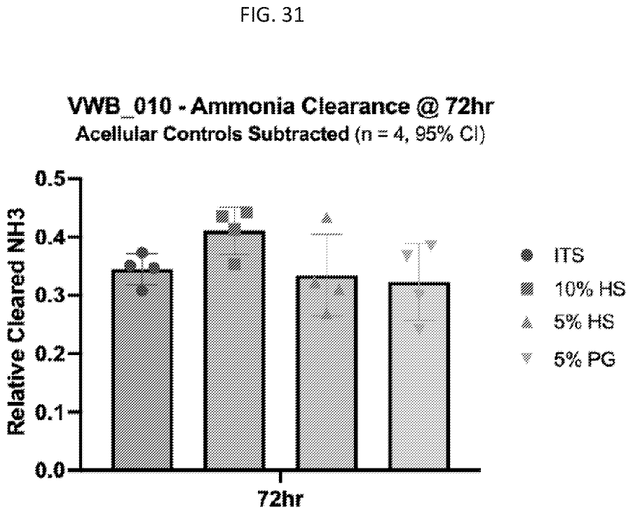

In terms of overall ammonia clearance at 72 hours, the ITS, 5% HS and 5% PG conditions performed similarly. The 10% HS condition had the highest level of overall relative ammonia clearance (FIG. 31).

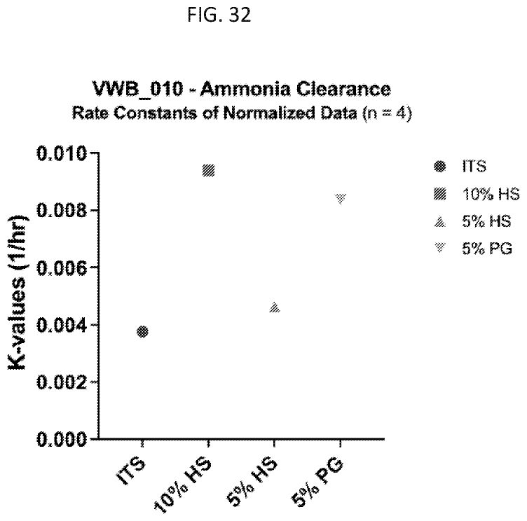

When looking at the k-values, the ITS condition had the lowest k-value, whereas the 10% HS condition had the highest k-value. The 10% HS condition showed the highest k-value, suggesting this condition cleared ammonia at a faster rate compared to the other groups (FIG. 32).

In summary, the 10% human serum condition yielded the lowest ALT/AST levels, the highest k-value, and most percent ammonia clearance in vitro. The 10% HS aggregation media generated comparable results with the control condition of ITS and outperformed the 5% HS and PG conditions. Based on the LDH analysis the 10% HS generated the least amount of total LDH/reactor during the aggregation process, while for the encapsulation step generated slightly better results compared to the control condition of ITS. Similar behavior was observed for the ALT/AST tests. The 10% HS had the highest relative ammonia clearance at 72 hours out of all groups.

### Example 2. Optimizing Cell Density, Ratio of Fibroblasts, and Culture Time

This study evaluated the effects of total cell density, the ratio of fibroblasts to hepatocytes, and duration of culture time as key process parameters, e.g., for scale up of the aggregation unit operation. The key outputs for optimization included aggregate (“seed”) quantity, mean seed diameter, seed viability, and seed potency. In addition to these key outputs, additional outputs of ALT/AST/LDH enzyme secretion were measured.

Experiments were performed to assess the parameters of cell density (total number of live cells/volume of culture of media; 3×10⁵ to 1.5×10⁶ viable cells/mL), the ratio of fibroblasts to hepatocytes (normal human dermal fibroblast (NHDF): primary human hepatocytes (PHH); 0.2-3.8), and culture time (0-70 hours aggregation). Table 3 illustrates the parameters that were tested.

TABLE 3
Experimental Group Information

| | Experiment | NHDF:PHH | Total Cell Density |
|---|---|---|---|
| Group | Day | Ratio | (cells/mL) |
| 1 | 1 | 0.96 | 553,590 |
| 2 | 1 | 0.96 | 1,246,410 |
| 3 | 1 | 3.8 | 900,000 |
| 4 | 1 | 2 | 300,000 |
| 5 (Control) | 1 | 2 | 900,000 |
| Reactors | 5 | | |
| VWB working volume | 85 | | |
| (mL) | | | |
| 6 | 2 | 3.04 | 553,590 |
| 7 | 2 | 3.04 | 1,246,410 |
| 8 | 2 | 0.2 | 900,000 |
| 9 | 2 | 2 | 1,500,000 |
| 10 (Control) | 2 | 2 | 900,000 |
| Reactors | 5 | | |

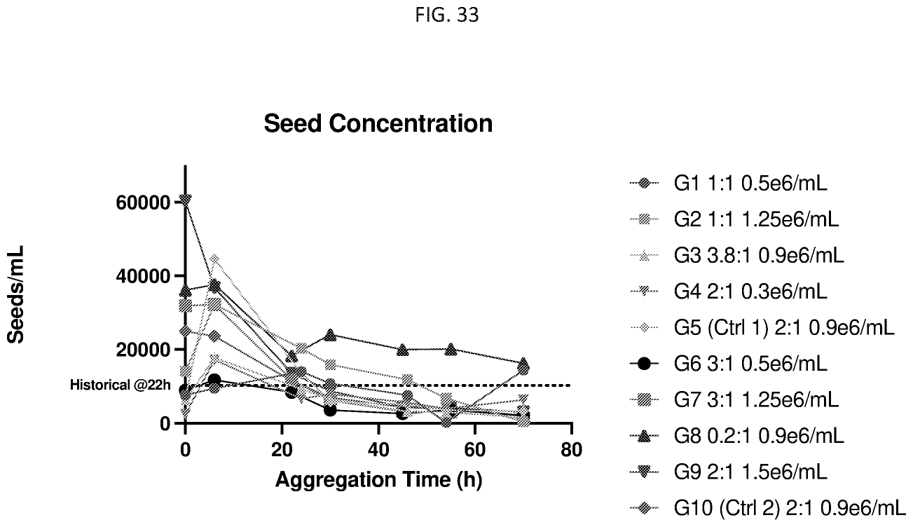

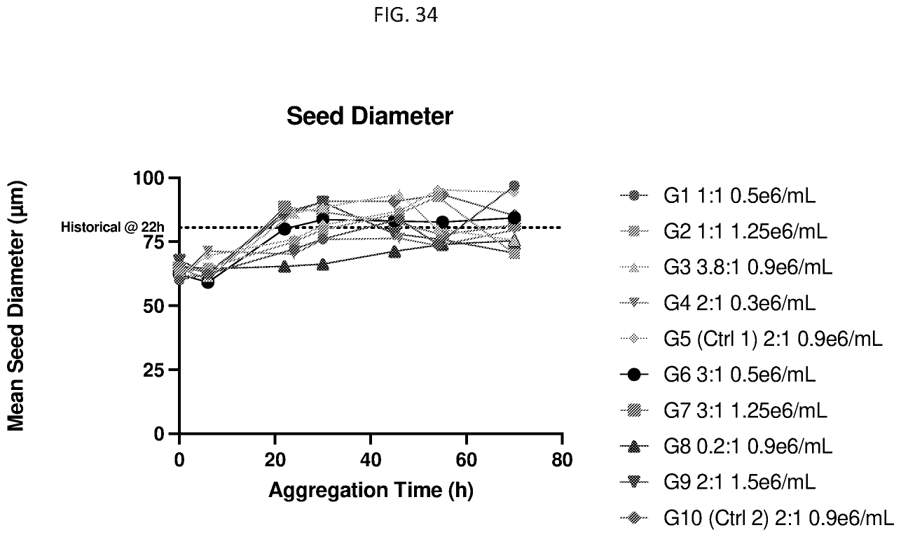

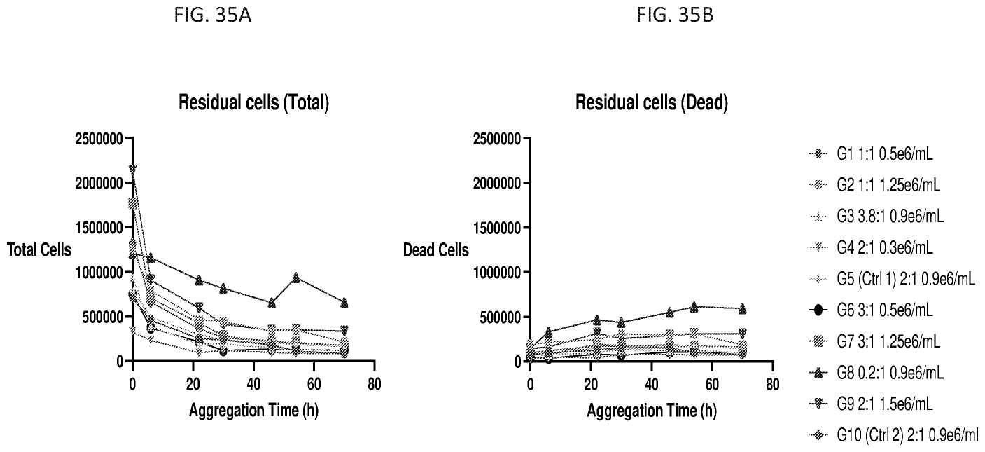

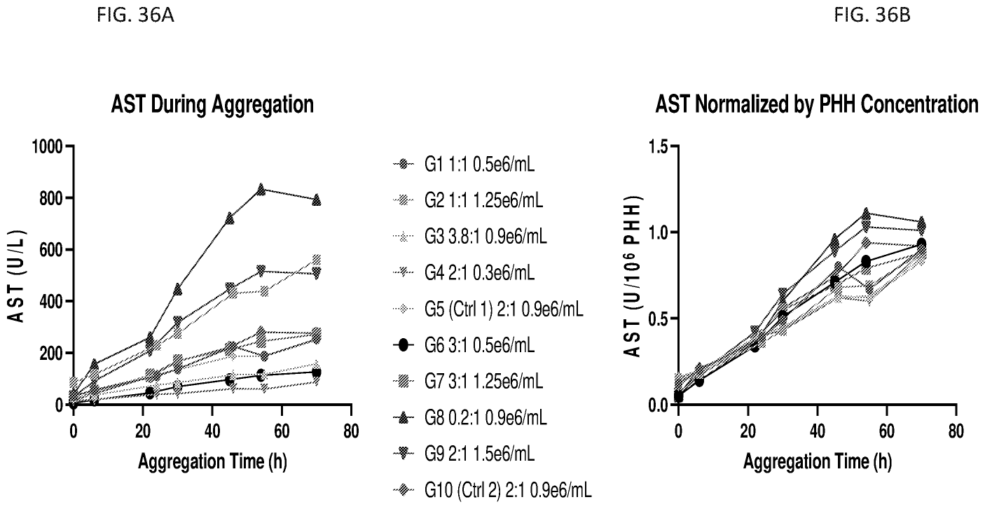

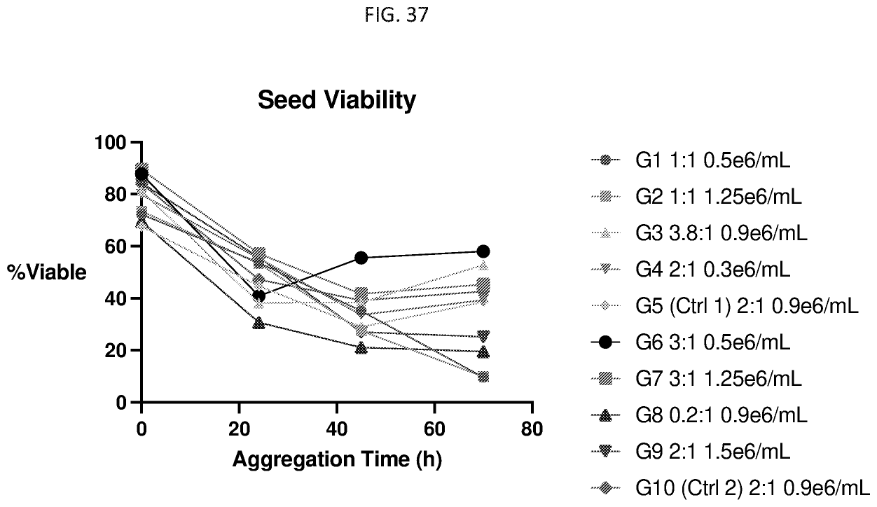

Key readouts included seed size, see count, seed potency, seed viability, and NHDF: PHH ratio. Results are shown in FIGS. 33-37.

In suspension, there are multiple modalities for scaling the seed generation process. Utilizing a design of experiments (DOE) approach, multiple parameters in a single study were able to be tested. Here a circumscribed central composite response surface design was employed. The design surface was repeated across several timepoints during aggregate formation in the vertical wheel bioreactors and consisted of 9 points across reactor seeding density and fibroblast-to-hepatocyte ratio, where the center point was repeated twice (group #5 and 10). Proprietary modelling software was used to optimize the model. Several optimums across multiple experiments were observed, with some patterns observed across the three major groups of responses.

### Viability

High hepatocyte-to-fibroblast ratio, low density, and low time were associated with maximal viability. This suggests a potential protective effect of the fibroblasts with hepatocyte-specific measurements of viability and may reflect a tendency for fibroblasts to survive better than hepatocytes when measured with global measurements of viability.

### Seed Morphology

The optimal surface location for morphology depended on the evaluated response. For seed size, medium values in ratio, density, and time produced maximum size. For seed volume fraction, i.e., seed yield, ratio was not a significant factor, and high density and low time produced higher seed volume fraction. The shorter time producing higher yield could be driven by a tendency to form super aggregates with more time. Indeed, for super aggregate formation, low ratio, medium density, and low time were best for minimal super aggregate formation. If normalized seed volume fraction was normalized by ALT, then the only difference from the standard seed volume fraction was that the normalized version for ALT preferred medium-high ratios, whereas the standard seed volume fraction was unaffected by ratio.

### Potency

Low ratio and high density maximized the raw potency (ammonia decay constant) per reactor volume, with no effect of time across t=22 or 46 hrs. When taking the ratio of potency per seed volume fraction, the optimum location was unchanged, except for a slight preference towards the later timepoint of t=46 hours. And when the ratio of potency per seed and super aggregate volume fraction was taken into consideration, the low ratio and later time was still preferred, except with an optimum at medium density (instead of high density). Taken together, the potency data suggest that lower ratios are associated with faster ammonia clearance, which could be due to a raw increase in hepatocytes per volume that are apparently uninhibited by metabolic constrains and overcomes the lower viability at lower ratios.

Overall, these results show that more hepatocytes yielded higher potency. A ratio of 0.2 NHDF:PHH and a density of 1.5 M cells/mL was productive in producing seeds using the methods described herein.

### Example 3. Encapsulation of Aggregates in a Graft

Satellite grafts were fabricated with seeds produced by microwells or vertical wheel bioreactors. Seeds for Groups 1, 3, and 4 were seeded in microwells. Seeds for Groups 2 and 5 were seeded in VWB bioreactors. Grafts were transported as single satellite grafts per 15 ml conical tube in a portable incubator with non-gas permeable caps. A total of 10 grafts per group were produced, and 2 acellular FIBRYGA® graft controls (52 total grafts).

Seeds were harvested from the VWB or microwell using a standard procedure, then centrifuged to form a master seed pellet. The master seed pellet was then resuspended in wash media at a projected concentration of 18 M PHH/mL and further aliquoted into sub-batches of 2 satellite grafts (160 μL volume). Then each sub-batch was quickly mixed with thrombin and dispensed into casting molds. The grafts were then allowed to polymerize fully at 37° C. in an incubator for 45 minutes. Transport media was added to the casting mold dish to hydrate the grafts during imaging. After imaging or after 15 minutes have elapsed since transport media addition, whichever was longer, the grafts were then manually transferred to transport tubes containing transport media, and then shipped via courier for surgical implantation into animals.

After the master seed mixture was transferred from the 50 ml conical tube to the sub-aliquot Eppendorf tubes, the initial 50 ml conical tube was retained and the appearance of the residual seeds on the surface of the conical tube was noted. The seeds generated by microwell aggregation appeared more irregular and clumpy in comparison to the VWB-generated seeds, which had a finer and more uniform appearance. The VWB seeds were more readily suspended homogeneously, which simplifies aliquoting into sub-batches. VWB seeds also appeared to have a longer settling time in wash media or fibrinogen, which is consistent with their smaller size.

### In Vitro Studies

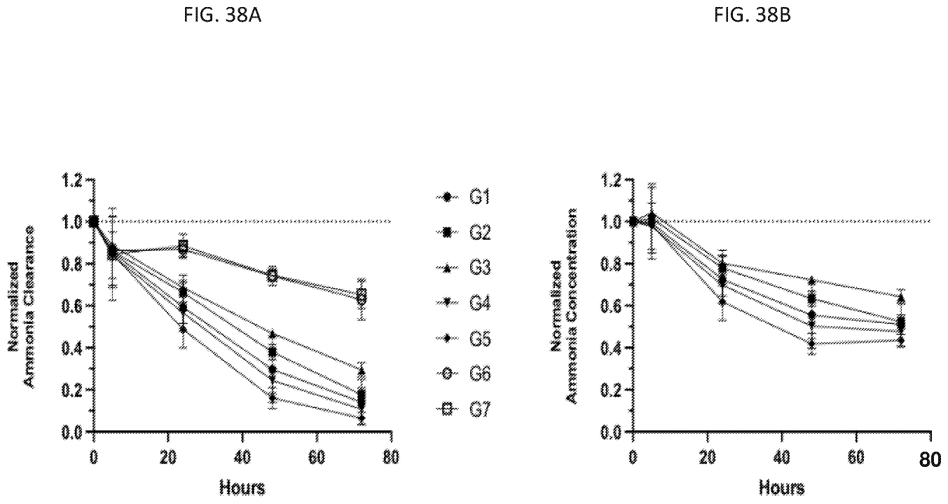

Two sub-batches of 60 uL of seed-fibrinogen suspension was produced and polymerized with thrombin (1:1 mixture as with the in vivo grafts) to form surrografts. Surrografts were then transferred to gas permeable plates (separated by experimental condition) and challenged with ammonium chloride. Samples were collected at hour 0 (i.e., 5 minutes after challenge), hour 5, hour 24, hour 48, and hour 72 to assess ammonia clearance. Two sets of surrograft controls were also assessed-Group 6: GMP housing, acellular; and Group 7: RUO housing, acellular. All groups had similar ammonia clearance levels to historical data (FIGS. 38A and 38B), and Group 3 had slightly less ammonia clearance, which was most obvious by hour 72.

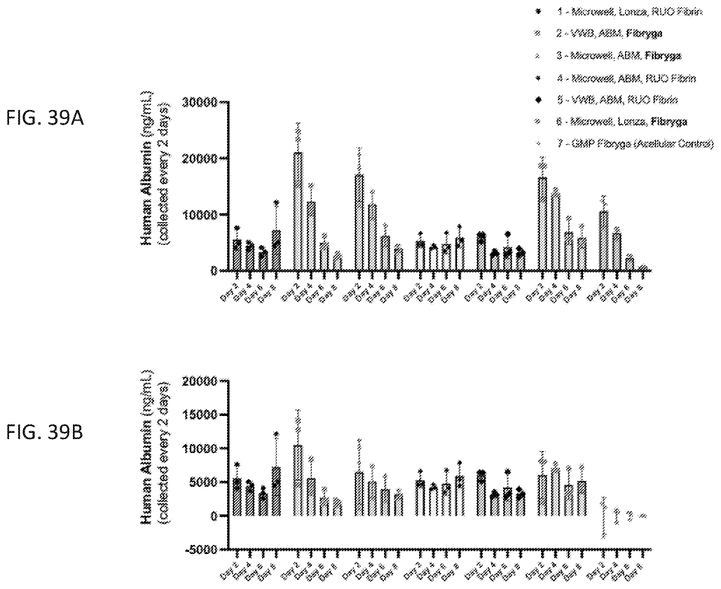

Surroseeds were prepared using the same seed-fibrinogen suspension as for in vivo grafts and in vitro surrografts but were diluted further with fibrinogen (matched to the original fibrinogen reagent) to a final concentration of 0.6 M PHH/mL upon formation of the final drug product. Grafts were cultured in 24-well plates for 8 days, with media exchanged and collected every 2 days. It was observed that human albumin signal detected in grafts formed with human-derived housing reagent at early timepoints was higher than in grafts formed with bovine-derived housing reagent (FIG. 39A). As part of the U1 surroseed execution, Group 6 (acellular FIBRYGA® graft) was used in an attempt to assess signal derived from the housing material alone. If it was assumed that albumin signal is a linear combination of the albumin signal from the cells and the albumin signal from the housing reagent, then one can subtract the day-matched albumin signal from Group 6 from the signal measured in Groups 2, 3, and 6 (since these were grafts formed with Fibryga). This pre-processing of the data was performed (FIG. 39B). All grafts exhibited detectable U1 surroseed biomarkers at all timepoints.

### In Vivo Studies

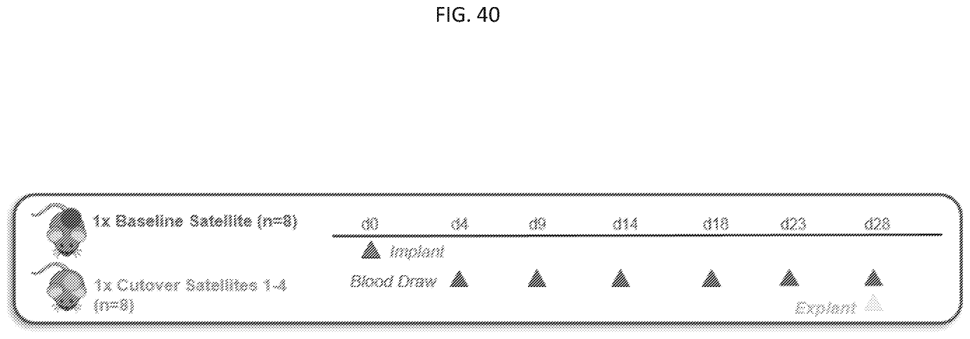

Grafts were implanted on Day 0, then whole blood was drawn and processed to plasma on days 4, 9, 14, 18, 23, and 28. Timepoints were chosen to satisfy 3× blood draws in a 2 week timeframe at minimum (FIG. 40).

### Biomarker Data—Albumin, Transferrin, and Correlation

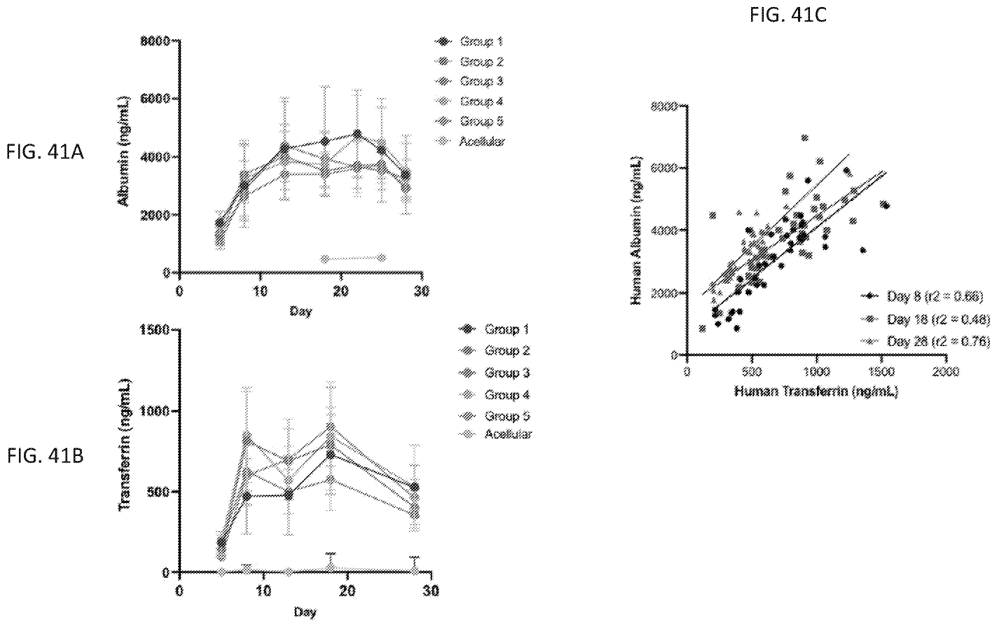

After implantation, the drug product integrates and the PHH secreted human proteins into the blood compartment. Whole blood was drawn and processed to plasma, then frozen. Samples were then thawed and analyzed via ELISA targeting human albumin (Bethyl), or human transferrin (Abcam, ab187391). The values for Groups 2 (new product/process changes) and the controls (Groups 3-5) were statistically similar to baseline (Group 1) and were sustainably produced through post-operative day 28 (FIG. 41A). Plasma biomarker levels for albumin and transferrin were plotted in a correlation plot (FIGS. 41B and 41C) and exhibited r-squared values suggesting that these biomarkers correlated with each other.

### Histopathology Review

All grafts were explanted on post-op day 28 and stored in NBF fixative for 24-30 hours at ambient temperature, then transferred to 70% histology-grade ethanol at ambient and then analyzed. Histological readouts were mostly consistent with readouts from prior studies explanted at the same timeline (POD 28-32), but higher degrees of fibrosis and dystrophic calcification were noted in some animals. Dystrophic calcification has been uncommon in other 9M/mL dose density-formulated grafts, though a possible explanation is that grafts in GMP matrix were noted to be softer and more flimsy than baseline formulation, which could lead to a higher incidence rate of graft folding and invagination within the graft bed, thus creating macro-structures that lead to an unideal metabolic environment. The degree of fibrosis appeared to be higher in GMP matrix vs research-grade matrix, and also appeared to be higher in VWB-aggregated seeds compared to microwell-aggregated seeds. Additionally, a prominence of non-PHH cellularity in the graft region was noted especially in grafts formed with the GMP matrix.

### 2D Vascularization Analysis

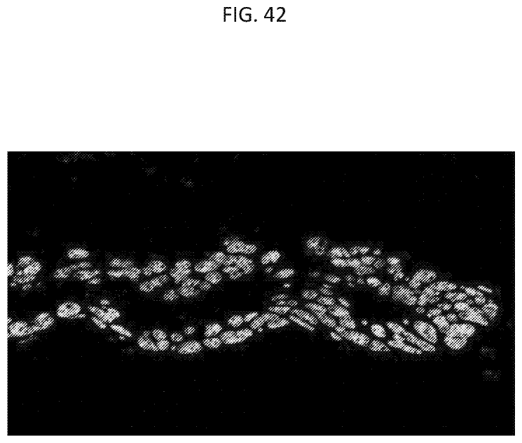

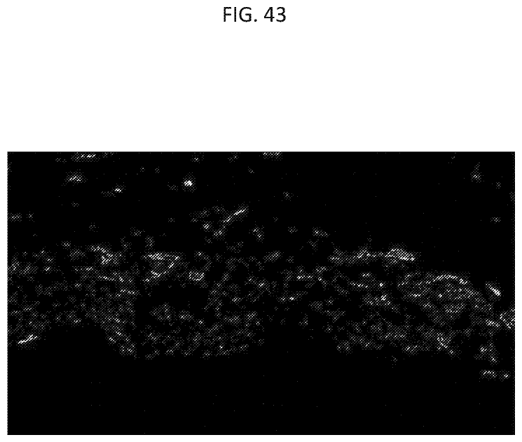

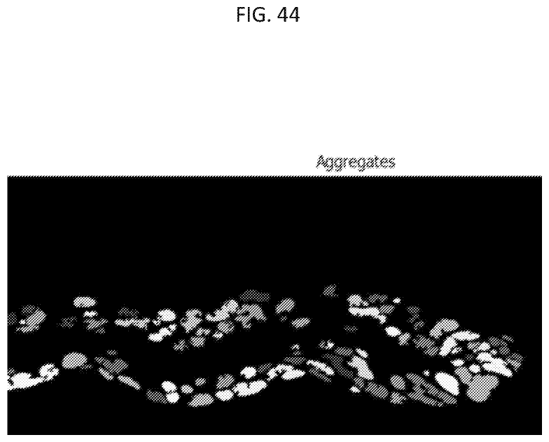

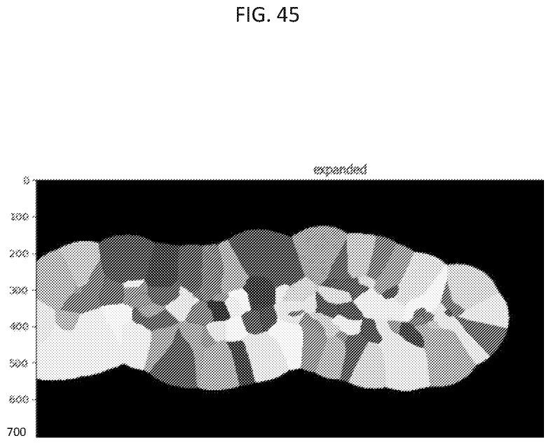

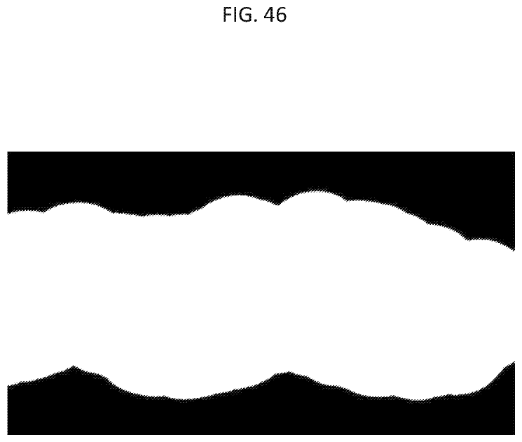

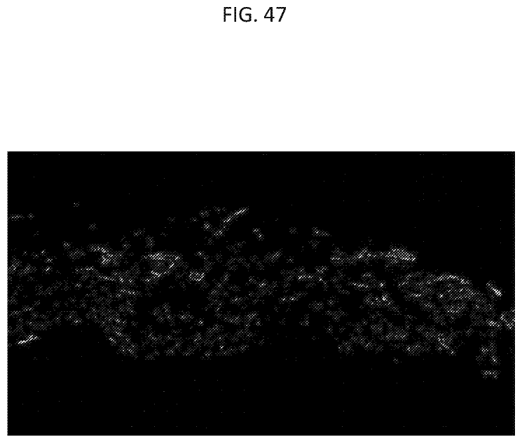

The level of vascularity was analyzed for each group using CK18-stained hepatocytes (FIG. 42) and CD31-stained vessels (FIG. 43). Images were acquired using OlyVIA and Cell Profiler was used to detect and count hepatocyte aggregates (FIG. 44). The aggregate areas were expanded (FIG. 45) to generate a mask to define the aggregate containing area (FIG. 46). This mask was then applied to the vascular image (FIG. 47). CD31 staining was used as the marker for vessels with Angiotool within the masked region to evaluate the vascular network associated with the hepatocyte region. Metrics such as average vessel length, number of hepatocytes per square millimeter, vessels per square millimeter, and vessels per hepatocyte aggregate were calculated from the output of Angiotool and the Cell Profiler analyses.

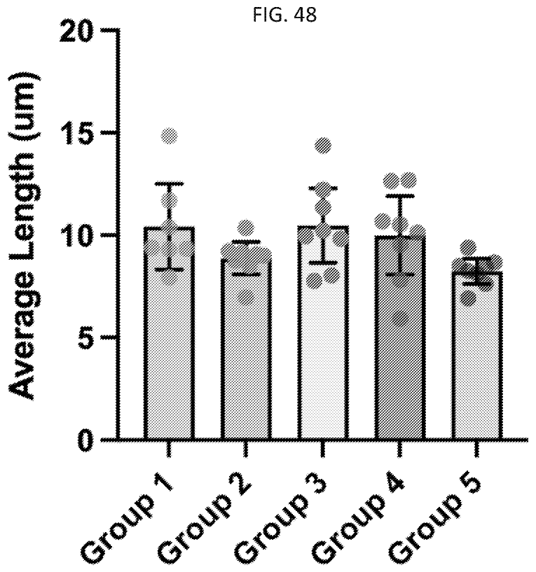

Analysis of average vessel length showed no significant difference between groups (FIG. 48). This analysis was restricted to the region surrounding the hepatocytes and did not include the hydrogel headspace.

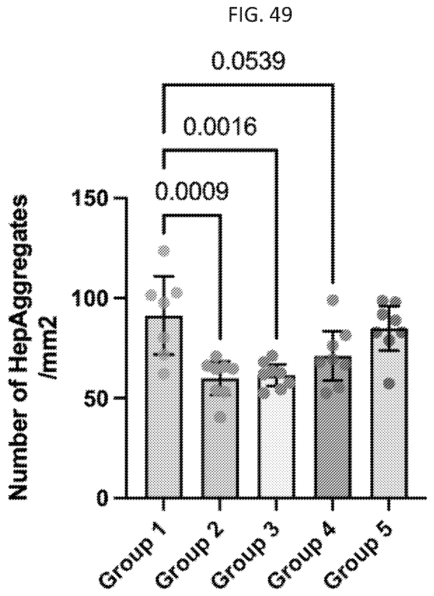

Analysis of hepatocyte aggregates per square millimeter showed a significant difference between group 1 vs. groups 2 and 3 and borderline significance with group 4 (FIG. 49). This could be driven by histology sampling or also by per-graft seed dosing. However, the seed pellet weight data showed that group 1 had the lowest seed pellet weight at 15% lower than the other groups, which is opposite from what was expected. All other groups exhibited similar seed pellet weights.

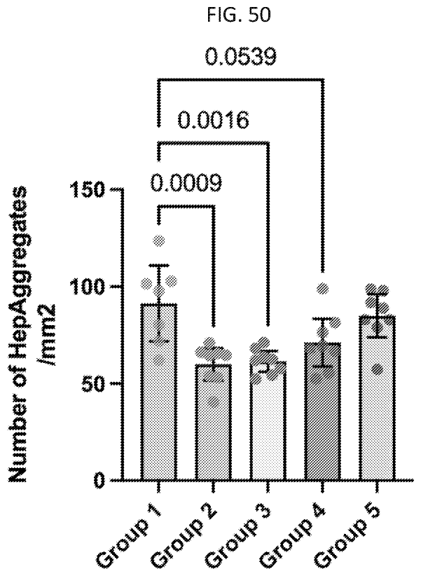

Analysis of the number of vessels per square millimeter showed a trend but not significant difference between groups 1 and 2 (p value >0.05) and a significant difference between groups 2 and 4 (FIG. 50). Groups 1 and 4 were similar in vessel density, but the variance in group 1 is likely the cause for lack of significance when compared to group 2. The difference between groups 1 and 4 was that ABM cells expanded in xenogeneic-free media and used in a thaw-and-aggregate format. This implies that use of those cells does not directly lead to a reduction in vessel density. But when those cells are used in VWB aggregates within the same hydrogel matrix (group 5) they yielded a lower vessel density. When those cells are used in microwell aggregates but in F1 hydrogel they yielded a lower vessel density. Both VWB aggregates and F1 hydrogel contributed to a lower vessel density, which is consistent with the additive effect observed in the lowest vessel density for Group 2. This analysis of vessels per unit area was limited to the region local to the hepatocytes and doesn't query the regions of hydrogel.

The vessel density may depend on factors like aggregate density as well as fibroblast health and density. The fact that groups 1 and 4 were equivalent rules out the new source and process for fibroblasts as a driver for reducing vessel density. The lower vessel density observed when the factors of VWB and F1 were isolated suggested that retention of fibroblasts within aggregates and the nature of the hydrogel could influence vessel density either directly or indirectly.

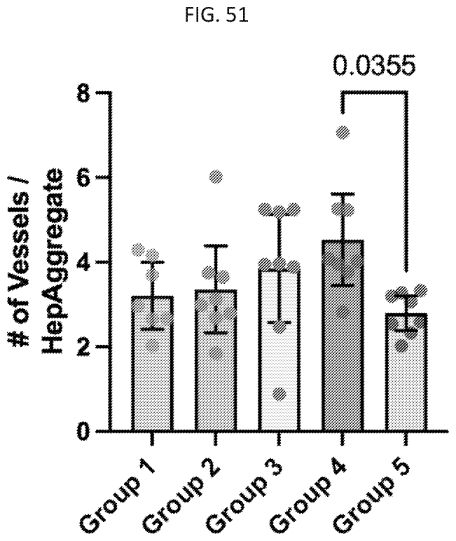

Analysis of the number of vessels per hepatocyte aggregate showed no significant difference except between groups 4 and 5 (FIG. 51). The difference between these groups is that group 4 was microwell aggregates and group 5 was vertical wheel aggregates, showing the microwell yielded higher vessels/aggregate. Groups 4 and 5 were in RUO fibrin. When VWB and microwell aggregates were compared in the background of GMP-grade F1 hydrogel the trend remains with VWB (group 2) being slightly lower than microwell (group 3) but on the background of F1 hydrogel the difference was not significant.

The metric of vessels per hepatocyte aggregate is the most robust metric to potential sources of sampling bias based on the fact that these data are from analysis of histology slices, which do not necessarily represent the entire graft. All groups had ˜3 vessels per hepatocyte aggregate.

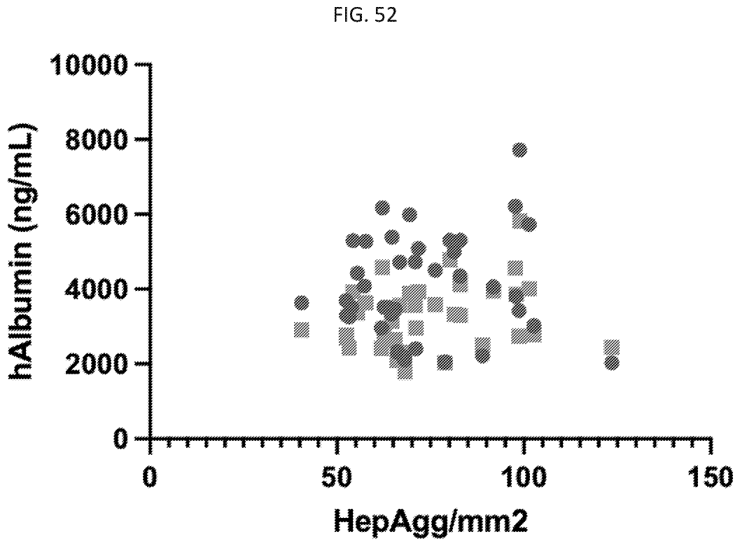

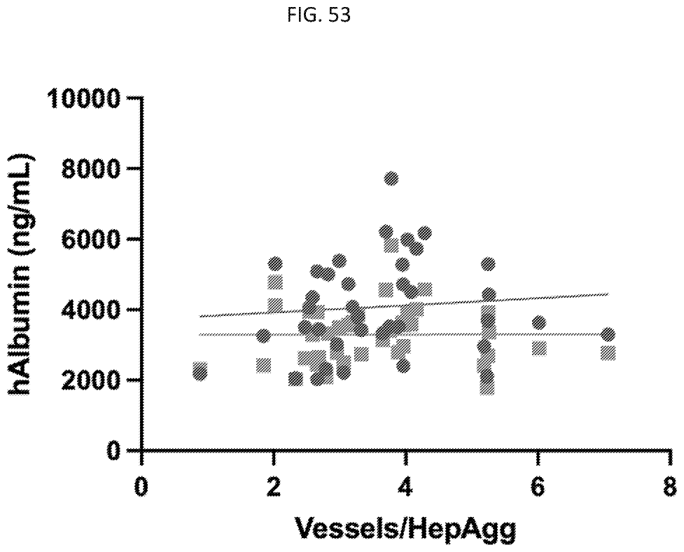

The data from the vascular analysis were evaluated for correlations with secreted albumin levels. Both the aggregate density (FIG. 52) and vessels per aggregate (FIG. 53) were evaluated and no correlation was observed. Given the linear correlation between albumin and transferrin, it was not expected that transferrin levels would have any correlation to these measures of vascularity either.

Overall, these data show that grafts produced with aggregates produced by the vertical wheel bioreactor were capable of being vascularized.

### Example 12. In Vitro Study for Measuring Seed Layer Height of Vertical Wheel Bioreactor Seeds

Due to the need to control seed layer height of the final graft for dose control, experiments assessing seed layer heights were performed. Vertical wheel bioreactor (VWB)-based seed layer heights trended differently than microwell based seed layer heights. It was assessed whether VWB seed layer heights generated from ROTEA™ washed NHDFs, PHHs, and seeds similar to non-ROTEA™ washed and/or microwell seed layer heights at dose densities of 3, 6, and 9 M/mL

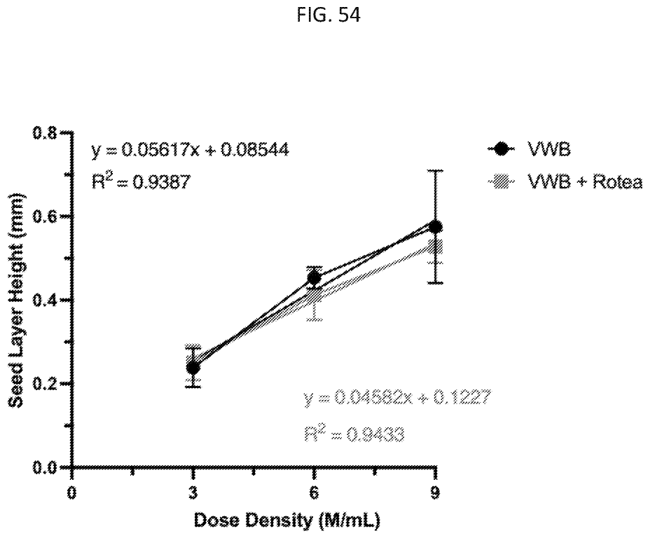

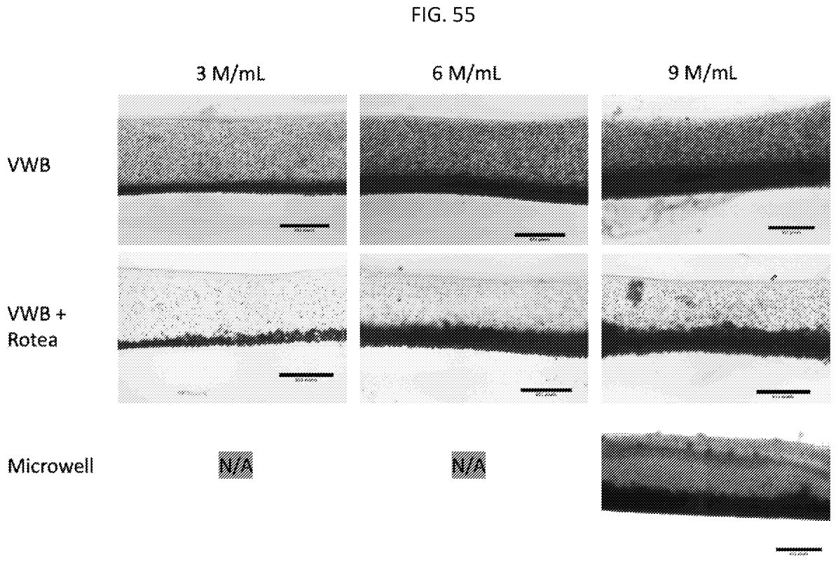

ROTEA™ washing NHDFs and PHHs post-thaw and seeds post-aggregation appears to yield on average slightly shorter seed layer heights compared to non-ROTEA™ washed seed layer heights at dose densities of 3, 6 and 9 M/mL (FIG. 54). T-test showed no significant difference between the two groups, indicating that the ROTEA™ washes did not impact the formation of seed layer heights. This also shows that the debris and seeds washed out during the ROTEA™ protocol were not the seeds that form the bulk of the seed layer height. This suggests that the ROTEA™ removed the cellular debris and smaller seeds that did not settle quick enough or the debris observed in the fibrin headspace in graft cross-sections. The clearer fibrin headspace allows for better distinguishing of the seed layer height as seen in FIG. 55.

In conclusion, these studies showed that ROTEA™ washed cells and seeds can be utilized without having a significant impact on seed layer height.

### Example 13. Scaling Bioreactor Production

Experiments were performed to assess seed health as a function of VWB fill volume. NHDFs and PHHs were thawed from cryogenic storage, counted, and seeded at designated concentrations in a 2 NHDF: 1 PHH ratio in PBS MINI 3×0.1 L bioreactors or 1×0.5 L bioreactor. Four different fill volumes were tested: 1) 60 mL, 2) 75 ml (control), 3) 100 mL and 4) 375 mL. The suspension cultures were allowed to run for 22 hours before harvest. After the 22 hours of aggregation the suspension cultures were harvested, sampled, and counted for analysis.

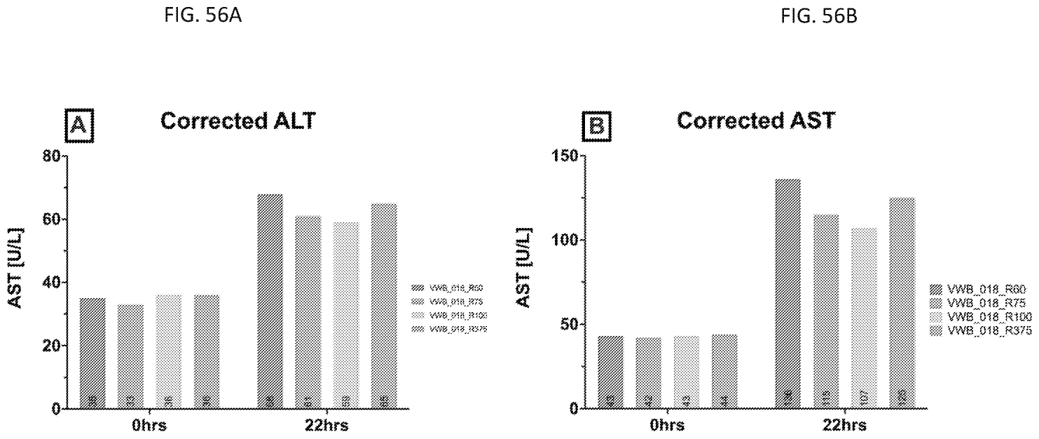

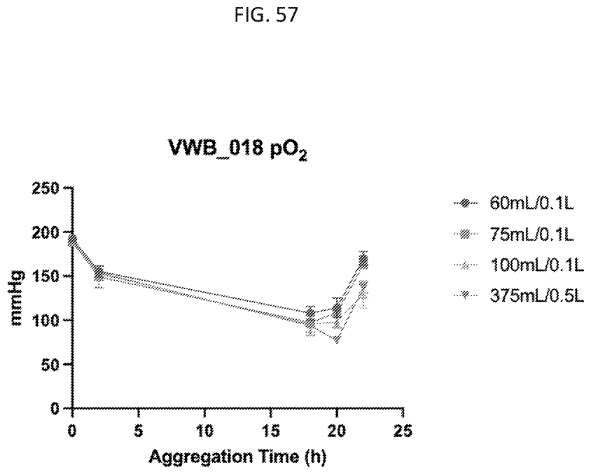

As shown in FIGS. 56A and 56B, ALT and AST levels remained constant between different fill volumes (60 mL, 75 mL, 100 mL, and 375 mL), indicating that cell health was not deleteriously affected during scale up of aggregation. Similarly, partial oxygen pressures remained similar between the four fill volumes (FIG. 57). Overall, these results suggest that the VWB system can be used to scale up bioreactor volumes for production of aggregates.

### Example 14. Maintaining Cell Numbers During Aggregation

Experiments were performed to show that the total number of cells does not substantially increase during aggregation, such that the methods promote aggregation and not expansion.

A population of PHHs and a population NHDFs were aggregated for 60 hours in suspension. Extracellular and total lactate dehydrogenase (LDH) was assessed at time points of 0 hours, 24 hours, and 60 hours. Cell viability was also assessed (Table 4).

TABLE 4
Viable cells during aggregation

| % viability | NHDF | PHH | NHDF + PHH |
|---|---|---|---|
| t = 0 hr | 100 | 70.03 | 82.4 |
| t = 24 hr | 96.2 | 49.3 | 61.9 |
| t = 60 hr | 92.6 | 28.0 | 45.5 |

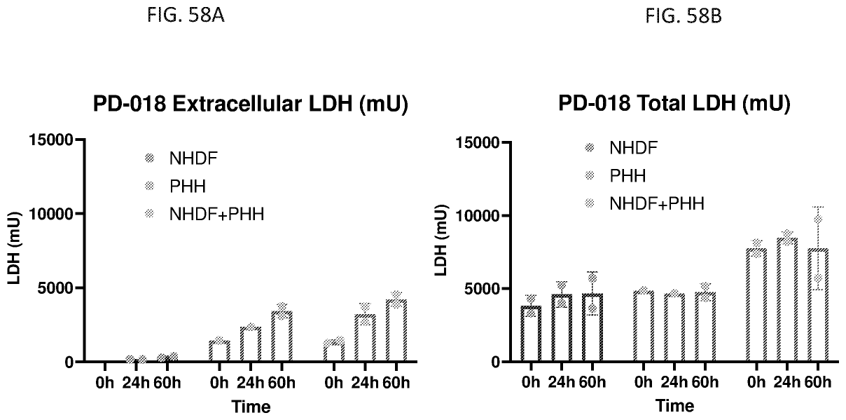

As shown in FIGS. 58A and 58B, extracellular LDH in the media accumulated over time. Total LDH remained constant over time, suggesting that the cells were not significantly proliferating. Taken together, this data suggests that over time, more cells were dying and that increased extracellular LDH was not due to an increase in the number of cells.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A method producing aggregates of a plurality of cell populations comprising a first population of cells and a second population of cells, the method comprising the steps of:
(a) agitating a liquid medium comprising the first population of cells and the second population of cells in a bioreactor for a duration sufficient to form aggregates comprising the first population of cells and the second population of cells, wherein the first population of cells and the second population of cells are in suspension in the liquid medium during the agitating step, thereby producing aggregates of a first population of cells and a second population of cells; and
(b) collecting the aggregates comprising the first population of cells and the second population of cells.

2. The method of claim 1, wherein the first population of cells comprises stromal cells.

3. The method of claim 2, wherein the stromal cells comprise fibroblasts.

4. The method of claim 3, wherein the fibroblasts are primary fibroblasts, induced pluripotent stem cell (iPSC)-derived fibroblasts, or embryonic stem cell (ESC)-derived fibroblasts.

5. The method of claim 3 or 4, wherein the fibroblasts are genetically engineered fibroblasts.

6. The method of any one of claims 1-5, wherein the second population of cells comprises parenchymal cells.

7. The method of claim 6, wherein the parenchymal cells comprise hepatocytes or hepatocyte precursor cells.

8. The method of claim 7, wherein the hepatocytes comprise primary human hepatocytes, iPSC-derived hepatocytes, or ESC-derived hepatocytes.

9. The method of claim 6 or 7, wherein the hepatocytes are genetically engineered hepatocytes.

10. The method of claim 6, wherein the parenchymal cells comprise pancreatic cells or pancreatic precursor cells.

11. The method of claim 10, wherein the pancreatic cells comprise alpha, beta, gamma, delta, or epsilon cells, or a combination thereof.

12. The method of claim 11, wherein the pancreatic cells comprise primary human pancreatic cells, iPSC-derived pancreatic cells, or ESC-derived pancreatic cells.

13. The method of any one of claims 10-12, wherein the pancreatic cells are genetically engineered pancreatic cells.

14. The method of claim 1, wherein the first population of cells comprises fibroblasts and the second population of cells comprises hepatocytes.

15. The method of claim 1, wherein the first population of cells comprises fibroblasts and the second population comprises hepatocyte precursor cells.

16. The method of claim 1, wherein the first population of cells comprises fibroblasts and the second population comprises pancreatic beta cells.

17. The method of any one of claims 1-16, wherein a ratio of the first population of cells to the second population of cells is from 10:1 to 1:10.

18. The method of claim 17, wherein the ratio is 2:1.

19. The method of any one of claims 1-18, wherein a ratio of the first population of cells to the second population of cells in the collected aggregates of step (b) is from 10:1 to 1:10.

20. The method of any one of claims 1-19, wherein the first population of cells are present at a density of 1×10⁴ cells/mL to 1×10⁸ cells/mL.

21. The method of claim 20, wherein the first population of cells comprises fibroblasts and the density of the fibroblasts is 1×10⁵ cells/mL to 1×10⁷ cells/mL.

22. The method of claim 21, wherein the density of the fibroblasts is 6×10⁵ cells/mL.

23. The method of any one of claims 1-22, wherein the second population of cells are present at a density of 1×10⁴ cells/mL to 1×10⁸ cells/mL.

24. The method of claim 23, wherein the second population of cells comprises hepatocytes and the density of the hepatocytes is 1×10⁵ cells/mL to 1×10⁷ cells/mL.

25. The method of claim 24, wherein the density of the hepatocytes is 3×10⁵ cells/mL.

26. The method of any one of claims 1-25, wherein the plurality of cell populations further comprises one or more additional cell populations.

27. The method of any one of claims 1-26, wherein the bioreactor has a capacity of 0.1 L to 500 L.

28. The method of claim 27, wherein the bioreactor has a capacity of 100 L to 300 L.

29. The method of any one of claims 1-28, wherein the first population of cells and the second population of cells do not expand by more than 30% during the agitating step.

30. The method of anyone of claims 1-29, wherein the bioreactor is a stir tank bioreactor or a vertical wheel bioreactor.

31. The method of any one of claims 1-30, wherein the bioreactor further comprises a bioprocess controller that controls one or more of pH, temperature, and dissolved oxygen concentration.

32. The method of any one of claims 1-31, wherein the first population of cells and the second population of cells do not adhere to the bioreactor.

33. The method of any one of claims 1-32, wherein at least 80% of the aggregates have a mean diameter±10% of each other.

34. The method of any one of claims 1-33, wherein the aggregates are spheroids.

35. The method of any one of claims 1-34, wherein the agitating step comprises a duration of from 1 hour to 72 hours.

36. The method of any one of claims 1-35, wherein the agitating step comprises a duration of up to 24 hours.

37. The method of claim 36, wherein the duration is up to 18 hours.

38. The method of any one of claims 1-37, wherein the agitation comprises rotating the bioreactor at a speed of 10 RPM to 50 RPM.

39. The method of any one of claims 1-38, wherein the medium has a viscosity of 0.9 cP to 1.4 cP.

40. The method of any one of claims 1-39, wherein the medium comprises 1-20 μg/mL recombinant human insulin, 1-10 μg/mL human transferrin, and 1×10⁻³ to 1×10⁻² μg/mL selenite.

41. The method of any one of claims 1-40, wherein the medium comprises laminin, collagen, elastin, or fibronectin.

42. The method of claim 41, wherein the medium comprises 1-10 μg/mL laminin.

43. The method of any one of claims 1-42, wherein the medium comprises 1-20 μM Rho-associated protein kinase (ROCK) inhibitor.

44. The method of claim 43, wherein the ROCK inhibitor is Y27632.

45. The method of any one of claims 1-44, wherein the medium comprises human serum.

46. The method of claim 45, wherein the medium comprises from 0.1% to 20% (v/v) human serum.

47. The method of claim 46, wherein the medium comprises 1% to 10% (v/v) human serum.

48. The method of claim 46, wherein the medium comprises 10% (v/v) human serum.

49. The method of any one of claims 1-48, wherein the medium comprises platelet lysate.

50. The method of claim 49, wherein the medium comprises 0.1% to 10% (v/v) platelet lysate.

51. The method of claim 50, wherein the medium comprises 1% to 5% (v/v) platelet lysate.

52. The method of any one of claims 1-51, wherein the medium comprises from 4,000 mg/L to 5,000 mg/L glucose.

53. The method of any one of claims 1-52, wherein the medium comprises from 10 ng/ml to 100 ng/ml glucagon.

54. The method of any one of claims 1-53, wherein the medium comprises fibrinogen.

55. The method of any one of claims 1-54, wherein the medium comprises from 10 ng/ml to 100 ng/ml dexamethasone.

56. The method of any one of claims 1-55, wherein the bioreactor is incubated at temperature of 35° C. to 39° C.

57. The method of any one of claims 1-56, wherein the method produces a density of aggregates of 500 aggregates/mL to 10,000 aggregates/mL.

58. The method of any one of claims 1-57, wherein the method produces an average aggregate mean diameter of from 50 μm to 200 μm.

59. The method of any one of claims 1-58, wherein the method produces an average total volume of aggregates of 200 μL to 50 mL.

60. The method of any one of claims 1-59, wherein the aggregates each comprise at least 103 cells.

61. The method of any one of claims 1-60, wherein the aggregates each have a mean diameter of at least 50 μm.

62. The method of any one of claims 1-61, wherein collecting the aggregates comprises pipetting, pouring, decanting, or draining the bioreactor.

63. The method of claim 62, wherein the bioreactor comprises a collection tube and the method comprises collecting the aggregates in the collection tube.

64. The method of any one of claims 1-63, further comprising washing the collected aggregates of step (b).

65. The method of any one of claims 1-64, further comprising purifying the collected aggregates of step (b).

66. The method of claim 65, wherein the collected aggregates are purified by centrifugation or acoustic separation.

67. The method of claim 66, wherein the centrifugation comprises counter flow centripetal centrifugation or density gradient centrifugation.

68. The method of any one of claims 65-67, wherein at least 80% of the aggregates have a mean diameter±10% of each other after purification.

69. The method of any one of claims 1-68, further comprising concentrating the collected aggregates of step (b).

70. The method of any one of claims 1-69, further comprising formulating the collected aggregates of step (b) in a storage buffer.

71. The method of any one of claims 1-70, further comprising washing the cells prior to step (a).

72. The method of any one of claims 1-71, further comprising the step of (c) encapsulating the collected aggregates of step (b) in a biocompatible scaffold.

73. A method encapsulating aggregates of a plurality of cell populations comprising a first population of cells and a second population of cells, the method comprising the steps of:
(a) agitating a liquid medium comprising the first population of cells and the second population of cells in a bioreactor for a duration sufficient to form aggregates comprising the first population of cells and the second population of cells, wherein the first population of cells and the second population of cells are in suspension in the liquid medium during the agitating step, thereby producing aggregates of a first population of cells and a second population of cells;
(b) collecting the aggregates of step (a); and
(c) encapsulating the aggregates of step (b) in a biocompatible scaffold.

74. The method of claim 72 or 73, wherein encapsulating the aggregates comprises providing a polymerizing agent or cross-linking reagent to polymerize or cross-link the biocompatible scaffold, thereby encapsulating the aggregates.

75. The method of claim any one of claims 72-74, wherein the biocompatible scaffold comprises fibrinogen.

76. The method of claim 74 or 75, wherein the polymerizing agent comprises thrombin.

77. The method of any one of claims 72-76, wherein the biocompatible scaffold further comprises a reinforcing agent.

78. The method of claim 77, wherein the reinforcing agent comprises collagen, poly (ethylene glycol), polyvinylidene acetate (PVDA), polyvinylidene fluoride (PVDF), poly (lactic-co-glycolic) acid (PLGA), or poly (I-lactic acid) (PLLA).

79. The method of any one of claims 73-78, further comprising washing the collected aggregates of step (b) prior to encapsulation.

80. The method of any one of claims 73-79, further comprising purifying the collected aggregates of step (b) prior to encapsulation.

81. The method of claim 80, wherein the collected aggregates are purified by centrifugation or acoustic separation.

82. The method of claim 81, wherein the centrifugation comprises counter flow centripetal centrifugation or density gradient centrifugation.

83. The method of any one of claims 80-82, wherein at least 80% of the aggregates have a mean diameter±10% of each other after purification.

84. The method of any one of claims 73-83, further comprising concentrating the collected aggregates of step (b) prior to encapsulation.

85. The method of any one of claims 73-84, further comprising formulating the collected aggregates of step (b) in a storage buffer prior to encapsulation.

86. The method of any one of claims 73-85, further comprising washing the cells prior to step (a).
